(19) United States
(12) Patent Application Publication
(10) Pub. No.: US 2024/0158788 A1
(43) Pub. Date: May 16, 2024

(54) METHODS AND COMPOSITIONS USED TO MODIFY CHROMATIN ARCHITECTURE TO REGULATE PHENOTYPE IN AGING AND CANCER

(71) Applicant: AGEX THERAPEUTICS, INC, Alameda, CA (US)

(72) Inventor: Michael D. West, Mill Valley, CA (US)

(21) Appl. No.: 18/279,963

(22) PCT Filed: Mar. 2, 2022
(86) PCT No.: PCT/US2022/018563

§ 371 Date: Sep. 1, 2023

Related U.S. Application Data
(60) Provisional application No. 63/274,734, filed on Nov. 2, 2021.
(60) Provisional application No. 63/155,626, filed on Mar. 2, 2021.

(51) Int. Cl. C12N 15/113; A61K 35/545; A61K 45/06; A61P 35/00

(52) U.S. Cl. CPC C12N 15/113; A61K 35/545; A61K 45/06; A61P 35/00; C12N 2310/14

(57) ABSTRACT

Compositions and methods are disclosed for modifying the chromatin structure of cells for therapeutic effect. More specifically, compositions and methods are disclosed to modify the relative expression of the nuclear lamins LMNA and LMNB1 to modify the regenerative potential of cells and to modify the heterogeneous states of cancer cells to improve therapeutic outcomes and thereby reduce tumor burden in diverse cancer types. The methods have application in veterinary and human medicine.

### RELATED APPLICATIONS

The instant application claims priority to U.S. Provisional Application No. 63/155,628, filed Mar. 2, 2021; and U.S. Provisional Application No. 63/274,734, filed Nov. 2, 2021; entire contents of each of which are expressly incorporated by reference herein in their entireties.

### FIELD OF THE INVENTION

The present invention relates to compositions and methods for the in vitro and in vivo delivery of factors that modulate the developmental age of cells and tissues. More specifically, methods and formulations are described that alter the three-dimensional architecture of the chromatin in cells by targeting heterochromatin and chromatin interactions with lamin A and lamin B1 which in turn modifies patterns of gene expression associated with embryonic-fetal and neonatal transitions for therapeutic effect. The therapeutic outcomes can be to increase the regenerative potential of adult mammalian tissues, or alternatively, to improve therapeutic outcomes in cancer therapy.

### BACKGROUND

Advances in stem cell technology, such as the isolation and propagation in vitro of human pluripotent stem (hPS) cells constitute an important new area of medical research. hPS cells have a demonstrated potential to be propagated in the undifferentiated state and then to be induced subsequently to differentiate into any and all of the cell types in the human body, including complex tissues. This has led, for example, to the prediction that many diseases resulting from the dysfunction of cells may be amenable to treatment by the administration of human embryonic stem cell-derived of various differentiated types (Thomson et al., Science 282:1145-1147 (1998)).

In regard to differentiating hPS cells into desired cell types, the potential to clonally isolate lines of human embryonic progenitor cells provide a means to propagate novel highly purified cell lineages with a prenatal, more specifically, a pre-fetal (embryonic) pattern of gene expression useful for regenerating tissues such as skin in a scarless manner. Such cell types have important applications in research, and for the manufacture of cell-based therapies (see PCT application Ser. No. PCT/US2006/013519; U.S. patent application Ser. No. 11/604,047; and U.S. patent application Ser. No. 12/504,630, each incorporated herein by reference in its entirety).

More recently, the potential of pluripotent stem cells and derived embryoid bodies for in vitro self-assembly into 3-dimensional organoids has generated interest as a potential pathway for both obtaining tissue for transplantation (Singh et al, Stem Cells Dev. 2015. 24(23): 2778-95) as well as modeling human embryonic development. In contrast to embryonic cells, fetal and adult-derived cells often show reduced potential for such organoid formation, organogenesis in vitro, and epimorphic regeneration in vivo. Epimorphic regeneration, sometimes referred to as “epimorphosis,” refers to a type of tissue regeneration wherein a blastema of relatively undifferentiated mesenchyme proliferates at the site of an injury followed by scarless regeneration of the original tissue histology.

The developmental timing of the loss of epimorphic potential cannot be fixed precisely, and likely varies with tissue type, nevertheless, the embryonic-fetal transition (EFT), or eight weeks of human development (Carnegie Stage 23; O'Rahilly, R., F. Müller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3):907-17). We previously disclosed that tissue regeneration, as opposed to scarring, reflects the presence of an embryonic as opposed to fetal or adult phenotype (see PCT Patent Application Ser. Nos. PCT/US2014/040601, PCT/US2017/036452, and PCT/US2020/025512, each of which is incorporated by reference herein in its entirety). Therefore, the term “embryonic” as used herein refers to the state of the differentiation of mammalian cells wherein the cell possess a scarless regenerative phenotype which therefore distinguishes them from that of the cells of the same differentiated type but in in a fetal or adult non-regenerative state of development that have little to no capacity for scarless regeneration. The term “embryonic” generally refers to development up to approximately Carnegie Stage 23, however, depending upon the tissue, may occur later in development. Excluded from the definition are cell types capable of scarless regeneration in the adult state such as hepatocytes and blood cells.

In the 1950s, experimental embryologists extended these studies to chick embryonic development. The regenerative nature of embryonic anlagen was widely utilized in experimental embryo transplant studies, such as the cross-transplantation of embryonic tissues from quail to chicken. Studies of disaggregated limb bud chondrogenic and myogenic cells as well as embryonic mesonephros and retina could re-associate and regenerate scarlessly, however, aggregation markedly decreased later in the course of development^{5,6}. This led to the question of the “why” and “how” of the developmental repression of cell-cell adhesion regulation during embryogenesis and the loss of regenerative potential.

In an attempt to answer the question as to “why” natural selection led to the repression of regeneration during development followed by aging, George Williams suggested the model of “antagonistic pleiotropy”, in part as an explanation of why “after a seemingly miraculous feat of morphogenesis a complex metazoan should be unable to perform the much simpler task of merely maintaining what is already formed⁷.” The theory posits that some traits selected for based on their value early in the reproductive period of the life cycle, have adverse effects in the post-reproductive years. One example is the nearly global repression of telomerase expression early in embryonic development and the re-expression of the gene (TERT) in ˜90% of cancer types⁸. The repression of TERT may reduce the risk of malignancy early in life, but may lead to cell senescence late in life.

It is commonly recognized that cancer cells and embryonic or fetal cells have properties in common. As early as 1859, based on histological comparisons, Rudolf Virchow proposed that cancers originated “by the same law that regulated the embryonic development.⁴³” Included among these common properties are an increased dependence on aerobic glycolysis (the Warburg Effect)⁴⁴. Indeed, it has been proposed that cancer, or cancer stem cells in general, are a result of failed embryonic cell maturation or reprogramming mature cells to an embryonic state⁴⁵.

Tumors, including the cancerous cells within them, are generally heterogeneous in regard to their behavior and responsiveness to therapy. One of the most important unsolved problem in oncology is to find more effective means of treating radio- or chemotherapy-resistant cancer. Following said therapies, relatively resistant cells arise that eventually lead to uncontrolled proliferation and metastasis. The resistant cells are often designated cancer stem cells (CSCs) and are widely believed to be more primitive, that is, more undifferentiated versions of cancer cells. Therefore, like many adult stem cells such as intestinal crypt stem cells or hematopoietic stem cells that rarely divide, CSCs are proposed to rarely divide and as a result are not targeted by many therapies designed to preferentially target proliferating cells.

In addition, cancer cells, in particular cancer cells of epithelial or glandular epithelial origin such as diverse carcinomas and adenocarcinomas are believed to be heterogeneous in regard to their status as epithelial or mesenchymal. It is currently widely believed that TGF beta signaling can transform epithelial cancer cells into a more mesenchymal state which in turn facilitates their migration or metastasis to distant sites followed by the original heterogeneity. Therefore, carcinomas and adenocarcinomas are believed to be capable of epithelial-mesenchymal transition (EMT) or mesenchymal-epithelial transition (MET). In addition, it is widely believed that cancer stem cells and carcinomas or adenocarcinomas that are in the mesenchymal state are one and the same thing. Given the importance of finding effective therapies to eradicate all cancer cells in humans and non-human mammals, defining the regulatory mechanisms behind this heterogeneity is of considerable relevance in understanding, diagnosing, and treating cancer.

In the present invention, we disclose the regulatory mechanism behind cancer cell heterogeneity in numerous cancer types including carcinomas, adenocarcinomas, sarcomas, gliomas, and blood cell cancers such as leukemias and lymphomas. We disclose that the consensus view that cancer stem cells are more primitive, that is to say, more undifferentiated, is the opposite of their true state. We disclose that cancer cells, such as carcinoma or adenocarcinoma cells displaying either cancer stem cell markers or EMT markers are in reality adult-like cells, while the pre-EMT cells more closely resembling the original epithelial cell in the case of carcinomas and adenocarcinomas, are embryo-like. Further, we disclose the role of nuclear lamins such as lamin A and lamin B1 in regulating the transition of diverse somatic cell types from the embryonic regenerative state to a non-regenerative fetal or adult-like state and in parallel, their role in controlling the generation of radio- and chemoresistant CSCs and the process of EMT. The disclosed compositions and methods expand our understanding of supposed cancer stem cells, with potential implications for cancer prognosis and therapy and have additional applications in inducing tissue regeneration in diverse aged tissue types as well as inducing maturation in embryonic cells and tissues.

We previously disclosed compositions and methods related to markers of the EFT in mammalian species and their use in modulating tissue regeneration (See, e.g. PCT Patent Application Ser. Nos. PCT/US2014/040601, PCT/US2017/036452, PCT/US2020/025512, and U.S. patent application Ser. No. 14/896,664, each of which is incorporated by reference herein in its entirety).

### SUMMARY

The present disclosure provides compounds, compositions, and methods useful for modifying the transition from an embryonic regenerative state to that of the non-regenerative fetal or adult states and vice versa in diverse mammalian somatic cells. The compositions and methods utilized to modify said developmental states are intended to alter the natural potential of regeneration in cells and tissues, to modify aging in said cells and tissues, and to alter cancer cells by inducing the maturation of cancer cells (induced cancer maturation (iCM) or reprogramming matured cancer cells back to an embryonic-like state to induce senolysis in cancer stem cells (iS-CSC). The compositions and methods are also directed to modifying the chromatin architecture of cells by modifying H3K9me3 methylation and/or by modifying the relative levels of nuclear lamin proteins lamin A and lamin B1 and/or or the transcription factors TCF3 or POU2F1. The compositions and methods of the present invention also are used for screening molecules for efficacy in modulating the embryonic or fetal/adult phenotype of cells, and for generating animal models for said research. The methods utilized in modifying gene expression in the present invention include methods of modifying regulatory noncoding RNAs and mRNAs involved in the embryonic-fetal transition (EFT) including gene therapy, RNA and miRNA-based therapy, and small molecule-based therapy.

Since we disclose herein that the increased expression of LMNA in cancer cells correlates with and regulates an adult-like phenotype (which is the CSC or EMT phenotype), reduction of levels of LMNA in cancer cells is a novel method of decreased metastasis in cancers. Said reduction can be accomplished by a variety of means such as RNAi.

According to one aspect, the present disclosure provides a method for inducing mammalian, including human tissue regeneration in a cell or tissue of a subject, comprised of the steps: 1) contacting the cell and/or tissue of the subject with an agent to induce a euchromatic state within heterochromatic regions of normal adult nonregenerative somatic cells; 2) contacting cells and/or tissue with factors capable of reversing developmental aging to restore an embryonic pattern of gene expression without inducing pluripotency, said factors including one or more of: OCT4, SOX2, KLF4, NANOG, ESRRB, NR5A2, CEBPA, MYC, SALL4, LIN28A, LIN28B and TERT.

In another aspect, the present disclosure provides a method is disclosed for inducing mammalian, including human tissue regeneration in a cell or tissue of a subject, comprised of the steps: 1) contacting the cell and/or tissue of the subject with an agent to induce a euchromatic state within the clustered protocadherin locus using a histone H3K9 methyltransferases inhibitor; 2) contacting cells and/or tissue with factors capable of reversing developmental aging to restore an embryonic pattern of gene expression without inducing pluripotency, said factors including one or more of: OCT4, SOX2, KLF4, NANOG, ESRRB, NR5A2, CEBPA, MYC, SALL4, LIN28A, LIN28B and TERT.

In another aspect, the present disclosure provides a method for inducing mammalian, including human tissue regeneration in a cell or tissue of a subject, comprised of the steps: 1) contacting the cell and/or tissue of the subject with an agent to induce a euchromatic state within the clustered protocadherin locus using a histone H3K9 methyltransferases inhibitor selected from the group consisting of one or more of SUV39H1, SUV39H2, and SETDB1; 2) contacting cells and/or tissue with factors capable of reversing developmental aging to restore an embryonic pattern of gene expression without inducing pluripotency, said factors including one or more of: OCT4, SOX2, KLF4, NANOG, ESRRB, NR5A2, CEBPA, MYC, SALL4, LIN28A, LIN28B and TERT.

In another aspect, the present disclosure provides a for inducing mammalian, including human tissue regeneration in a cell or tissue of a subject, comprised of the steps: 1) contacting the cell and/or tissue of the subject with an agent to induce a euchromatic state within the clustered protocadherin locus using a histone H3K9 methyltransferases inhibitor, wherein the histone H3K9 methyltransferases inhibitor in siRNA; 2) contacting cells and/or tissue with factors capable of reversing developmental aging to restore an embryonic pattern of gene expression without inducing pluripotency, said factors including one or more of: OCT4, SOX2, KLF4, NANOG, ESRRB, NR5A2, CEBPA, MYC, SALL4, LIN28A, LIN28B and TERT.

In some embodiments, a vector comprises a sequence encoding the siRNA. In some embodiments, the vector is a plasmid. In some embodiments, the vector is a viral vector. In some embodiments, the viral vector is an adeno-associated viral vector.

In another aspect, the present disclosure provides a method of inducing cancer maturation (iCM) in cancer cells in a subject, the method comprising: 1) obtaining a biological sample comprising cancer cells from the subject; 2) administering one or more agents to the cancer cells that alter the lamin A and/or lamin B1 expression from that of an embryonic state to that of a fetal or adult state, thereby inducing iCM.

In another aspect, the present disclosure provides a method of induce senolysis in cancer stem cells (iS-CSC) in cancer cells in a subject, the method comprising: 1) obtaining a biological sample comprising cancer cells from the subject; 2) administering one or more agents to the cancer cells that alter the lamin A and/or lamin B1 expression from that of an embryonic state to that of a fetal or adult state, thereby inducing iS-CSC.

In another aspect of the present disclosure, a method is disclosed for inducing mammalian, including human tissue regeneration in a cell or tissue of a subject, comprised of the steps: 1) contacting the cell and/or tissue of the subject with an agent to induce a euchromatic state within the clustered protocadherin locus using a histone H3K9 methyltransferase inhibitors; 2) contacting the cell and/or tissue with chemical iTR inducers, said iTR inducers selected from the group consisting of inhibitors of glycogen synthase 3 (GSK3), inhibitors of TGF-beta signaling, HDAC inhibitors, inhibitors of H3K4/9 histone demethylase LSD1, inhibitors of Dot1L, inhibitors of G9a, inhibitors of Ezh2, inhibitors of DNA methyltransferase, activators of 3′ phosphoinositide-dependent kinase 1, promoters of glycolysis, RAR agonists, agents that mimic hypoxia, activators of telomerase, inhibitors of the MAPK/ERK pathway, or combination thereof, thereby reverting fetal or adult-derived cells to their embryonic counterpart without reverting the cells in said tissue to pluripotent stem cells. In some embodiments, steps 1 and 2 are in vitro. In some embodiments, steps 1 and 2 are in vivo. In some embodiments, the iTR inducers include those capable in other conditions of improving the efficiency of inducing pluripotency in somatic cell types, that is, in generating iPS cells.

In another aspect of the present disclosure, a method is disclosed for inducing mammalian, including human tissue regeneration in a cell or tissue of a subject, comprised of the steps: 1) contacting the cell and/or tissue of the subject with an agent to induce a euchromatic state within the clustered protocadherin locus using a histone H3K9 methyltransferase inhibitors; 2) contacting the cell and/or tissue with one or more nucleic acids encoding TERT; and contacting the cell and/or tissue with chemical iTR inducers, said iTR inducers selected from the group consisting of combinations of inhibitors of glycogen synthase 3 (GSK3), inhibitors of TGF-beta signaling, HDAC inhibitors, inhibitors of H3K4/9 histone demethylase LSD1, inhibitors of Dot1L, inhibitors of G9a, inhibitors of Ezh2, inhibitors of DNA methyltransferase, activators of 3′ phosphoinositide-dependent kinase 1, promoters of glycolysis, RAR agonists, agents that mimic hypoxia, activators of telomerase, inhibitors of the MAPK/ERK pathway, or combinations thereof, thereby reverting fetal or adult-derived cells to their embryonic counterpart without reverting the cells in said tissue to pluripotent stem cells. In some embodiments, steps 1 and 2 are in vitro. In some embodiments, steps 1 and 2 are in vivo. In some embodiments, the inducers include those capable in other conditions of improving the efficiency of inducing pluripotency in somatic cell types, that is, in generating iPS cells.

In another aspect, the present disclosure provides a method for inducing tissue regeneration, comprised of the steps: 1) contacting the target cells with an agent that increases the ratio of lamin B1 protein compared to lamin A protein in the target cells; 2) contacting the target cells with one or more nucleic acids encoding TERT, wherein TERT transiently expressed to increase telomere length in the target cells. In some embodiments, the nucleic acids encoding TERT are in a plasmid or vector.

In some embodiments, the agent that increasing the ratio of lamin B1 protein compared to lamin A protein is one or more nucleic acids encoding LMNB1. In some embodiments, the agent that increasing the ratio of lamin B1 protein compared to lamin A protein is an siRNA targeting LMNA gene.

In some embodiments, the cells are mammalian. In some embodiments, the cells are human cells. In some embodiments, the cells are non-human mammalian cells.

In another aspect, the present disclosure provides a method of inducing senolysis in cancer stem cells of a subject, comprised of the steps: 1) contacting the cancer cells with an agent that increases the ratio of lamin B1 protein compared to lamin A protein in the cancer cells; 2) contacting to cancer cells with an agent that induces apoptosis in cells with DNA damage.

In some embodiments, the apoptosis-inducing agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, BSI-201, or a combination thereof.

In some embodiments, the agent that increases the ratio of lamin B1 protein compared to lamin A protein is one or more nucleic acids encoding TCF3. In some embodiments, the agent that increasing the ratio of lamin B1 protein compared to lamin A protein is an siRNA targeting LMNA gene.

In some embodiments, the cells are mammalian. In some embodiments, the cells are human cells. In some embodiments, the cells are non-human mammalian cells.

In another aspect, the present disclosure provides a method of inducing senolysis in cancer stem cells of a subject, comprised of the steps: 1) contacting the cancer stem cells iPSC with one or more reprogramming factors selected from OCT4, SOX2, KLF4, NANOG, ESRRB, NR5A2, CEBPA, MYC, SALL4, LIN28A, and LIN28B without reprogramming the cells to pluripotency, wherein the expression of the one or more reprogramming factors is transient; 2) contacting to cancer cells with an agent that induces apoptosis in cells with DNA damage.

In some embodiments, the reprogramming factors are SOX2, OCT4, and KLF4.

In some embodiments, the reprogramming factors are LIN28B, SOX2, NANOG, and OCT4.

In some embodiments, the apoptosis-inducing agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, BSI-201, or a combination thereof.

In some embodiments, the cells are mammalian. In some embodiments, the cells are human cells. In some embodiments, the cells are non-human mammalian cells.

In another aspect, the present disclosure provides a method of inducing senolysis in cancer stem cells of a subject, comprised of the steps: 1) contacting the cancer stem cells iPSC with one or more reprogramming factors selected

In another aspect, the disclosure provides methods of modifying the nuclear architecture in microbiopsies ex vivo to restore them to a state wherein they are capable of regenerating tissue scarlessly when transplanted.

In another aspect, the disclosure provides methods of modifying the modifying the nuclear architecture in vivo to restore them to a state wherein they are capable of participating in iTR.

In another aspect of the invention, methods are provided to cause iTR in tissues afflicted with degenerative disease including, but not limited to age-related disease wherein the means of effecting iTR in the diseased tissue utilizes a gene expression vector or vectors that cause the modified nuclear architecture disclosed herein including but not limited to altering the levels of the proteins encoded by the genes LMNA, LMNB1, POU2F1, TCF3, OCT4, SOX2, KLF4, NANOG, ESRRB, NR5A2, CEBPA, MYC, SALL4, LIN28A, and LIN28B together with telomerase catalytic component, such as human TERT.

In another aspect of the invention, methods are provided to cause iTR in tissues afflicted with degenerative disease including, but not limited to age-related disease and cancer wherein the means of effecting iTR in the diseased tissue utilizes a gene expression vector or vectors that cause the exogenous expression of the genes regulating the altered nuclear architecture associated with the EFT and EMT disclosed herein including but not limited to LMNB1, POU2F1, TCF3, OCT4, SOX2, KLF4, NANOG, ESRRB, NR5A2, CEBPA, MYC, SALL4, LIN28A, and LIN28B together with telomerase catalytic component, such as human TERT.

In another aspect of the invention, methods are provided to cause iTR in tissues afflicted with degenerative disease including, but not limited to age-related disease and cancer wherein the means of effecting iTR in the diseased tissue utilizes a gene expression vector or vectors that inhibit the expression of LMNA together with telomerase catalytic component, such as human TERT.

In another aspect, the disclosure provides a method of identifying a candidate modulator of lamin A and lamin B1 levels in a cell comprising: (a) the candidate modulator or multiplicity of modulators of said LMNA and LMNB1 levels in a purified state or in a mixture with other molecules; (b) somatic cells exhibiting a fetal or adult pattern of gene expression as opposed to an embryonic pattern of lamin gene expression; (c) a reporter construct present within the somatic cells or within extracts of said cells incapable of otherwise expressing embryonic lamin expression of interest wherein the promoter of a gene differentially regulated in somatic cells in the embryonic phases of development compared to fetal and adult stages drives the expression of a reporter gene; and (ii) determining whether the candidate modulator or a multiplicity of modulators affect expression of the reporter gene, wherein altered expression of the reporter gene as compared with expression of the gene in the absence of the candidate modulator indicates that the compound modulates the isoform activity to resemble embryonic expression.

In another aspect, the disclosure provides a method of identifying a candidate modulator of lamin A and lamin B1 levels comprising: (a) the candidate modulator or multiplicity of modulators of said lamin A and/or lamin B1 levels in a purified state or in a mixture with other molecules; (b) somatic cells exhibiting an embryonic pattern of lamin gene expression of interest as opposed to an adult pattern of gene expression; (c) a reporter construct present within the somatic cells or within extracts of said cells incapable of otherwise expressing adult lamin A wherein the promoter of the gene is differentially regulated in somatic cells in the embryonic phases of development compared to fetal and adult stages drives the expression of a reporter gene; and (ii) determining whether the candidate modulator or a multiplicity of modulators affect expression of the reporter gene, wherein altered expression of the reporter gene as compared with expression of the gene in the absence of the candidate modulator indicates that the compound modulates the level to resemble adult expression.

In some embodiments, a method of identifying a candidate modulator of lamin A and/or lamin B1 expression further comprises administering a candidate compound or multiplicity of compounds identified as modulators of lamin expression to a subject.

In some embodiments, a method of identifying a candidate global modulator of lamin A and/or lamin B1 expression further comprises administering a candidate compound for induced tissue regeneration to cells derived from fetal or adult sources and assaying the expression lamin A and/or lamin B1 expression through the use of an easily measured readout such as fluorescence generated from GFP driven by the promoter of said gene.

In some embodiments, a method of identifying a compound further comprises administering the compound to a subject. In some embodiments, the subject is a non-human animal, e.g., a non-human animal that serves as a model for tissue regeneration, wound healing, or cancer. In some embodiments, the subject is a human.

In another aspect, the present disclosure provides a pharmaceutical composition comprising: (a) a modulator of lamin A or lamin B1 expression; and (b) a pharmaceutically acceptable carrier.

In another aspect lamin A and/or lamin B1 expression are altered in cancer cells such that cancer cells expressing an embryonic phenotype are changed to that of a fetal or adult state to cause iCM.

In another aspect lamin B1 expression is reduced in cancer cells such that cancer cells expressing an embryonic phenotype are changed to that of a fetal or adult state to cause iCM.

In another aspect lamin B1 expression is reduced in cancer cells by the use of RNAi such that cancer cells expressing an embryonic phenotype are changed to that of a fetal or adult state to cause iCM.

In another aspect, genes regulating lamin A and/or lamin B1 expression are altered such that cancer cells are treated with agents that alter the expression of the genes from that of an embryonic state to that of a fetal or adult state to cause iCM.

In another aspect, genes regulating lamin A and/or lamin B1 expression are altered such that cancer cells are treated with agents that alter the expression of the genes from that of a fetal or adult state to that of an embryonic state to cause iS-CSC.

In another aspect, the present invention provides a means of engineering an animal model, preferably a mouse model capable of robust regenerative potential, said mouse being in a common laboratory strain of mice thereby facilitating molecular genetics and animal preclinical studies. Said robustly regenerating mouse is produced by creating mice that express either inducibly in all tissues or select tissues, or constitutively expressing various combinations of the genes LMNA, LMNB1, TCF3, and POU2F1 wherein said mice and breeding said mice together, provide for mouse models of regeneration, aging, and cancer.

### BRIEF DESCRIPTION OF THE DRAWINGS

The patent or application file contains at least one drawing executed in color. Copies of this patent or patent application publication with color drawing(s) will be provided to the Office upon request and payment of the necessary fee.

FIG. 1 depicts a schematic of the embryo-onco phenotype. Diagram shows the interactions of epithelium and underlying stromal cells (shown as fibroblastic cells) in embryonic tissue capable of regeneration (left panel), fetal-adult tissue capable only of scar-based wound healing (middle panel), and the twin phenotypes exhibited by malignant cancer cells wherein the cancer cells can display an embryonic pattern of gene expression such as when they are in the epithelial state (de-matured or “DC cells”) or alternatively, an adult-like state (adult cells “AC”) the latter previously referred to in the art inaccurately as “cancer stem cells”. Also illustrated is the predominant lamin expression in each state together with associated gene expression leading to cancer cell phenotypes.

FIG. 2 depicts a time course of LMNA RNA expression during development. LMNA expression by RNA-sequencing in FPKM is shown in cultured pluripotent stem cells (both hES and hiPS cells), fetal cells derived from the medial aspect of the upper arm of fetal skin spanning 8 weeks to 16 weeks of development, adult cells derived from the medial aspect of the upper arm of fetal skin spanning 11 years to 83 years of age (Neonatal-Old Age), In vitro-aged dermal fibroblast at early passage (Young) or at senescence (Senesc) in both quiescence (0.5% FBS) and in active log growth conditions (10% FBS). HGPS fibroblasts and age-matched controls, and adult-derived fibroblasts together with iPSC counterpart cell line. Fibroblasts unless otherwise noted are synchronized in quiescence (0.5% FBS). *s have common meaning, i.e. ns=non-significant, *=p<0.05, **=p<0.01, ***=p<0.001, ****=p<0.0001

FIG. 3 depicts a time course of LMNB1 RNA expression during development. LMNB1 expression by RNA-sequencing in FPKM is shown in cultured pluripotent stem cells (both hES and hiPS cells), fetal cells derived from the medial aspect of the upper arm of fetal skin spanning 8 weeks to 16 weeks of development, adult cells derived from the medial aspect of the upper arm of fetal skin spanning 11 years to 83 years of age (Neonatal-Old Age), In vitro-aged dermal fibroblast at early passage (Young) or at senescence (Senesc) in both quiescence (0.5% FBS) and in active log growth conditions (10% FBS). HGPS fibroblasts and age-matched controls, and adult-derived fibroblasts together with iPSC counterpart cell line. Fibroblasts unless otherwise noted are synchronized in quiescence (0.5% FBS). *s have common meaning, i.e. ns=non-significant, *=p<0.05, **=p<0.01, ***=p<0.001, ****=p<0.0001

FIG. 4 depicts a correlation matric showing correlation of pluripotency, embryonic, adult, epithelial, mesenchymal, and cancer stem cell markers in carcinomas. Diverse markers of pluripotent stem cells, embryonic (pre-fetal) cells, fetal and adult cells (Adult), markers of epithelium, and mesenchymal cells, and putative cancer stem cell markers are shown from 593 carcinoma cell lines illustrating the correlation of adult-like markers with mesenchymal cells and “cancer stem cell” markers.

FIG. 5 depicts a correlation matrix showing correlation of pluripotency, embryonic, adult, epithelial, mesenchymal, and cancer stem cell markers in sarcomas. Diverse markers of pluripotent stem cells, embryonic (pre-fetal) cells, fetal and adult cells (Adult), markers of epithelium, and mesenchymal cells, and putative cancer stem cell markers are shown in 43 sarcoma cell lines illustrating the correlation of adult-like markers with mesenchymal cells and “cancer stem cell” markers.

FIG. 6 depicts a correlation matrix showing correlation of pluripotency, embryonic, adult, epithelial, mesenchymal, and cancer stem cell markers in lymphoma cell lines. Diverse markers of pluripotent stem cells, embryonic (pre-fetal) cells, fetal and adult cells (Adult), markers of epithelium, and mesenchymal cells, and putative cancer stem cell markers are shown in 113 lymphoma cell lines illustrating the correlation of adult-like markers with mesenchymal cells and “cancer stem cell” markers.

FIG. 7 depicts a time course of LMNA RNA expression during reprogramming. LMNA expression compared to the pluripotency marker DNMT3B is shown for independent replicates of human iPS cell lines (hiPS Cells); adult-derived dermal fibroblasts before treatment and 3, 7, 11, 15, 20, 28, 35, 42, and 49 days after reprogramming with KLF4, OCT4, SOX2, and MYC; human embryonic stem cells (hESCs); and differentiated progeny of iPSCs.

FIG. 8 depicts a time course of LMNB1 RNA expression during reprogramming. LMNB1 expression compared to the pluripotency marker DNMT3B is shown for independent replicates of human iPS cell lines (hiPS Cells); adult-derived dermal fibroblasts before treatment and 3, 7, 11, 15, 20, 28, 35, 42, and 49 days after reprogramming with KLF4, OCT4, SOX2, and MYC; human embryonic stem cells (hESCs); and differentiated progeny of iPSCs.

FIG. 9 depicts a time course of TCF3 RNA expression during reprogramming. TCF3 expression compared to the pluripotency marker DNMT3B is shown for independent replicates of human iPS cell lines (hiPS Cells); adult-derived dermal fibroblasts before treatment and 3, 7, 11, 15, 20, 28, 35, 42, and 49 days after reprogramming with KLF4, OCT4, SOX2, and MYC; human embryonic stem cells (hESCs); and differentiated progeny of iPSCs.

FIG. 10 depicts a time course of POU2F1 RNA expression during reprogramming. POU2F1 expression compared to the pluripotency marker DNMT3B is shown for independent replicates of human iPS cell lines (hiPS Cells); adult-derived dermal fibroblasts before treatment and 3, 7, 11, 15, 20, 28, 35, 42, and 49 days after reprogramming with KLF4, OCT4, SOX2, and MYC; human embryonic stem cells (hESCs); and differentiated progeny of iPSCs.

FIGS. 11A-11B depicts a correlation of COX7A1 with adult cells and diverse cancer cells displaying CSC (EMT) markers. FIG. 11A RNA-sequence showing COX7A1 expression in pluripotent stem cells (hES and hiPS cells), diverse clonal hESC-derived embryonic progenitors, diverse fetal and adult-derived somatic cell types from all three germ layers, diverse adult-derived epithelial cells, diverse sarcoma cell lines, diverse carcinoma cell lines, and blood cancer cells. FIG. 11B Graph of COX7A1 expression by RNA-sequencing in 1018 diverse carcinoma, adenocarcinoma, sarcoma, blood cell cancers, gliomas, melanomas, as well as other cancer cell types sorted by expression of the EMT marker COL1A1.

FIGS. 12A-12B depicts a correlation of PCDHGA12 with adult cells and diverse cancer cells displaying CSC (EMT) markers. FIG. 12A. RNA-sequence showing PCDHGA12 expression in pluripotent stem cells (hES and hiPS cells), diverse clonal hESC-derived embryonic progenitors, diverse fetal and adult-derived somatic cell types from all three germ layers, diverse adult-derived epithelial cells, diverse sarcoma cell lines, diverse carcinoma cell lines, and blood cancer cells. FIG. 12B. Graph of PCDHGA12 expression by RNA-sequencing in 1018 diverse carcinoma, adenocarcinoma, sarcoma, blood cell cancers, gliomas, melanomas, as well as other cancer cell types sorted by expression of the EMT marker COL1A1.

FIG. 13 depicts the effects of scrambled knockdown constructs vs LMNA knockdown constructs on the proliferation (left) and migration (right) of the cancer cell line HT1080.

### DETAILED DESCRIPTION

### Abbreviations

AC—Adult-derived cells
AEC—Adult epithelial cells
AMH—Anti-Mullerian Hormone
ANE—Adult non-epithelial cells
ASC—Adult stem cells
ATAC-seq—Transposase-Accessile Chromnathin foowed by sequencing
CAC—Carcinoma and adenocarcinoma cells
CAR T-Cells—Chimeric antigen receptor modified T-cells
cGMP—Current Good Manufacturing Processes
CIMP—CpG Island Methylator Phenotype
CIMP-E—CpG Island Methylator Phenotype refers to the unique DMRs of hypermethylated sites in embryonic cells compared to their fetal and adult counterparts
CM—Cancer Maturation
CNS—Central Nervous System
CpG—CpG dinucleotide
CPL—Clustered protocadherin locus
DMEM—Dulbecco's modified Eagle's medium
DMR—Differentially-methylated region
DMSO—Dimethyl sulphoxide
DNAm—Changes in the methylation of DNA that provide a marker or “clock” of the age of cells and tissue.
DPBS—Dulbecco's Phosphate Buffered Saline
ED Cells—Embryo-derived cells; hED cells are human ED cells
EDTA—Ethylenediamine tetraacetic acid
EFT—Embryonic-Fetal Transition
EPs—Embryonic progenitor cells
ES Cells—Embryonic stem cells; hES cells are human ES cells
ESC—Embryonic Stem Cells
EVs—Extracellular Vesicles
FACS—Fluorescence activated cell sorting
FBS—Fetal bovine serum
FCs—Fetal cells
FPKM—Fragments Per Kilobase of transcript per Million mapped reads from RNA sequencing.
GFP—Green fluorescent protein
GMP—Good Manufacturing Practices
HAEC—Human Aortic Endothelial Cell
hEC Cells—Human Embryonal Carcinoma Cells
hED Cells—Human embryo-derived cells
hEG Cells—“Human embryonic germ cells” are stem cells derived from the primordial germ cells of fetal tissue.
hEP cells—human embryonic progenitor cells
hES cells—human Embryonic Stem Cells including human ES-like cells, therefore “hES cells or “hESCs) as used herein refer to both primed and naïve pluripotent stem cells.
HGPS—Hutchinson-Gilford Progeria Syndrome
hiPS Cells—“Human induced pluripotent stem cells” are cells with properties similar to hES cells obtained from somatic cells after exposure to hES-specific transcription factors such as SOX2, KLF4, OCT4, MYC, or NANOG, LIN28, OCT4, and SOX2 or other means that restore aged somatic differentiated cells to pluripotency.
hPS Cells—human pluripotent stem cells such as hES cells, hiPS cells, EC cells, and human parthenogenic stem cells.
HSE—“Human skin equivalents” are mixtures of cells and biological or synthetic matrices manufactured for testing purposes or for therapeutic application in promoting wound repair.
iCM—Induced Cancer Maturation.
iPS Cells—“Induced pluripotent stem cells” are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B or other factors capable of reversing the developmental aging of differentiated cells back to a pluripotent stem cell state essentially matching the gene expression profile of hES cells.
iS-CSC—“Induced Senolysis of Cancer Stem Cells” refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.
iTM—Induced Tissue Maturation
iTR—Induced Tissue Regeneration
MEM—Minimal essential medium
MSC—Mesenchymal stem cell
NCs—Neuronal cells, such as the cells of the CNS and peripheral nervous systems including neurons and glial cells such as astocytes and oligodendrocytes.
NT—Neonatal Transition
PBS—Phosphate buffered saline
PCs—Pluripotent stem cells
PCDH—Protocadherin
PCR—Polymerase Chain Reaction
PS fibroblasts—“Pre-scarring fibroblasts” are fibroblasts derived from the skin of early gestational skin or derived from ED cells that display a prenatal pattern of gene expression in that they promote the rapid healing of dermal wounds without scar formation.
PT—Pluripotency Transition
qRT-PCR—quantitative Real-Time PCR
RFU—Relative Fluorescence Units
RNAi—RNA Interference
RNA-seq—RNA sequencing
SC—Sarcoma Cells
SFM—Serum-Free Medium
siRNA—Small interfering RNA
St. Dev.—Standard Deviation
TR—Tissue Regeneration

### Definitions

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. patent application Ser. No. 10/304,020; PCT Patent Application Ser. No. PCT/US02/37899, PCT/US06/30632, each of which is incorporated by reference herein in its entirety).

The term “blastomere/morula cells” refers to blastomere or morula cells in a mammalian embryo or blastomere or morula cells cultured in vitro with or without additional cells including differentiated derivatives of those cells.

The term “cancer maturation” refers to the alteration of gene expression in premalignant or malignant cancer cells such that said premalignant or malignant cancer cells that initially express markers of embryonic cells, are altered to express markers of fetal or adult cells.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “cellular reconstitution” refers to the transfer of a nucleus of chromatin to cellular cytoplasm so as to obtain a functional cell.

The term “clonal” refers to a population of cells obtained the expansion of a single cell into a population of cells all derived from that original single cells and not containing other cells.

The term “clonal embryonic progenitor cells” refers to embryonic progenitor cells that derived in vitro from a single cell.

The term “cytoplasmic bleb” refers to the cytoplasm of a cell bound by an intact or permeabilized but otherwise intact plasma membrane, but lacking a nucleus.

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term “embryonic” as used herein refers to the state of the differentiation of mammalian cells wherein the cell possess a scarless regenerative phenotype which therefore distinguishes them from that of the cells of the same differentiated type but in in a fetal or adult non-regenerative state of development that have little to no capacity for scarless regeneration. The term “embryonic” generally refers to development up to approximately Carnegie Stage 23, however, depending upon the tissue, may occur later in development. Excluded from the definition are cell types capable of scarless regeneration in the adult state such as hepatocytes and blood cells. In the case of the human species, the transition from embryonic to fetal development occurs at about 8 weeks of prenatal development, in mouse it occurs on or about 16 days, and in the rat species, at approximately 17.5 days post coitum. (see, the web site: www.php.med.unsw.edu.au/embryology/index.php?title=Mouse_Timeline_Detailed).

The term “embryonic pattern of CPL isoform expression” refers to a pattern of gene expression characterized by activation of members of the α and β clusters and repression of members of the γ locus with the exception of PCDHGB4 and PCDHGB6 which are relatively highly expressed in embryonic cells.

The term “fetal-adult pattern of CPL isoform expression” or “adult pattern of CPL isoform expression” refers to a pattern of gene expression characterized by activation of expression of members of the α and β clusters and decreased expression of members of the γ locus with the exception of PCDHGB4 and PCDHGB6 which are relatively highly expressed in embryonic cells.

The term “embryonic progenitor cells” refers to cells of all somatic cell lineages that are more differentiated than pluripotent stem cells (e.g. embryonic stem cells) but have not matured so as to express markers of fetal or adult cell types. In the case of human embryonic progenitor cells, they would express markers of cells of less than eight weeks of gestation, such as relatively low to no expression of COX7A1 compared to fetal or adult-derived cells.

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

The term “global modulator of TR” or “global modulator of iTR” refers to agents capable of modulating a multiplicity of iTR genes or iTM genes including, but not limited to, agents capable of downregulating COX7A1 while simultaneously up-regulating PCDHB2, or down-regulating NAALADL1 while simultaneously up-regulating AMH in cells derived from fetal or adult sources and are capable of inducing a pattern of gene expression leading to increased scarless tissue regeneration in response to tissue damage or degenerative disease.

The term “human embryonic stem cells” (hES cells) refers to human ES cells.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced Cancer Maturation” refers to methods resulting in a change in the phenotype of premalignant or malignant cells such that subsequent to said induction, the cells express markers normally expressed in that cell type in fetal or adult stages of development as opposed to the embryonic stages. The term as used herein, is synonymous with epithelial-mesenchymal transition (EMT) when applied carcinomas and adenocarcinomas.

The term “induced tissue regeneration” refers to the use of the methods of the present invention as well as the methods disclosed in PCT Patent Application Ser. No. PCT/US2014/040601 and U.S. patent application Ser. No. 14/896,664 to alter the molecular composition of fetal or adult mammalian cells such that said cells are capable or regenerating functional tissue following damage to that tissue wherein said regeneration would not be the normal outcome in animals of that species.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iCM factors” refers to molecules that alter the levels of CM activators and CM inhibitors in a manner leading to CM in a tumor for therapeutic effect.

The term “iCM genes” refers to genes that when altered in expression can cause CM in a tumor for therapeutic effect.

The term “iTR factors” refers to molecules that alter the levels of TR activators and TR inhibitors in a manner leading to TR in a tissue not naturally capable of TR.

The term “iTR genes” refers to genes that when altered in expression can cause induced tissue regeneration in tissues not normally capable of such regeneration.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs.

In some embodiments a nucleic acid comprises standard nucleosides (abbreviated A, G, C, T, U). In other embodiments a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid is modified. Modifications may be located uniformly or nonuniformly, and the location of the modifications (e.g., near the middle, near or at the ends, alternating, etc.) can be selected to enhance desired propert(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided.

“Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc.

The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the invention encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the invention provides embodiments relating to precursor polypeptides and embodiments relating to mature versions of a polypeptide.

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The terms “small interfering RNA” (siRNA) and “RNA interference” (RNAi) are used interchangeably and otherwise consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 21 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long.

MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the invention. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors.

In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precursors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, a small molecule is non-polymeric. In some embodiments, a small molecule is not an amino acid. In some embodiments, a small molecule is not a nucleotide. In some embodiments, a small molecule is not a saccharide.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound of the invention).

The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present invention would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “TR activator genes” refers to genes whose lack of expression in fetal and adult cells but whose expression in embryonic phases of development facilitate TR.

The term “TR inhibitor genes” refers to genes whose expression in fetal and adult animals inhibit TR.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant invention is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

In some embodiments, the cells are further treated with radiation and/or a chemotherapeutic agent. In some embodiments, radiation and/or a chemotherapeutic agent can induce apoptosis when a cancer cell has DNA damage.

In some embodiments, the chemotherapeutic agent is a DNA damaging agent, checkpoint inhibitor, antibody, alkylating agent, antimetabolites, anthracyclines, nitrosoureas, topisomerase inhibitor, isomerase inhibitor, mitotic inhibitor, tyrosine kinase inhibitors, protease inhibitor, or a combination thereof.

In some embodiments, the DNA damaging agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, BSI-201, or a combination thereof.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain.

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a 6.times.His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the invention a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the invention relate to variants of a polypeptide, it will be understood that polynucleotides encoding the variant are provided.

The term “vector” is used herein to refer to a nucleic acid or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference in their entirety to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Surprisingly, numerous aspects of aging and age-related disease are taught in the present invention to be addressable by modifying the nuclear architecture as disclosed herein. This breadth of application reflects the pan-tissue alteration in expression associated with the loss of regeneration during development and oncogenesis. These manifestations of aging include age-related vascular dysfunction including peripheral vascular, coronary, and cerebrovascular disease; musculoskeletal disorders including osteoarthritis, intervertebral disc degeneration, bone fractures, tendon and ligament tears, and limb regeneration; neurological disorders including stroke and spinal cord injuries; muscular disorders including muscular dystrophy, sarcopenia, myocardial infarction, and heart failure; endocrine disorders including Type I diabetes, Addison's disease, hypothyroidism, and pituitary insufficiency; digestive disorders including pancreatic exocrine insufficiency; ocular disorders including macular degeneration, retinitis pigmentosa, and neural retinal degeneration disorders; dermatological conditions including skin burns, lacerations, surgical incisions, alopecia, graying of hair, and skin aging; pulmonary disorders including emphysema and interstitial fibrosis of the lung; and auditory disorders including hearing loss.

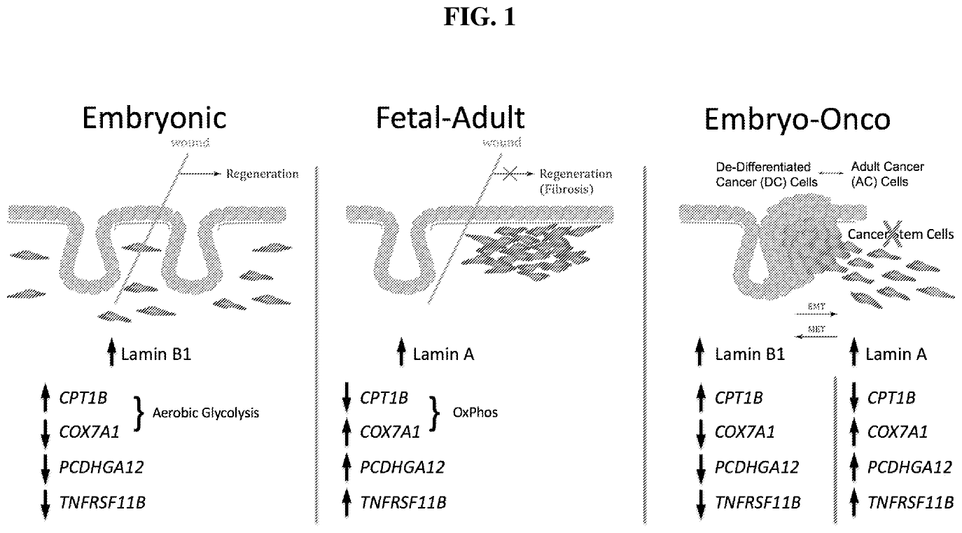

The present invention discloses novel methods of modulating the nuclear, more specifically, chromatin architecture of diverse somatic cells to shift the phenotype of cells from that of an embryonic to an adult phenotype or alternatively, from an adult phenotype to an embryonic one. As shown in FIG. 1, the present invention teaches that diverse somatic embryonic cells (corresponding to cells that have not progressed past the developmental stage of the embryonic-fetal transition (EFT)) relatively overexpress lamin B1 encoded by the gene LMNB1 compared to lamin A (encoded by the gene LMNA). The present invention teaches that a high LMNB1/LMNA expression ratio on a protein or mRNA level lead to a state of low COX7A1 expression and relatively high CPT1B expression (leading to aerobic glycolysis), relatively low to absent PCDHGA12, and TNFRS11B expression leading to increased proliferation and sensitivity to apoptosis. Said increased sensitivity to apoptosis may result in senolysis of cells with genotoxic damage commonly present in cancer cells and senescent cells.

Also illustrated in FIG. 1 is a depiction of normal fetal or adult diverse somatic cells that no longer are capable of scarless regeneration and instead repair tissue damage with a fibrotic response. The figure illustrates the present invention in that the relatively high ratio of LMNA/LMNB1 leads to decreased CPT1B and increased COX7A1 expression leading to increased oxidative phosphorylation, and increased PCDHGA12, TNFRSF11B, and increased TGF-beta signaling mediated in part by secreted factors such as those encoded by the genes: S100A3, S100A6, S199A10, S100A11, and S100A13.

Also illustrated in FIG. 1 is a depiction of the biphasic heterogeneous nature of cancer cells. We previously disclosed that cancer cells often display an embryonic pattern of gene expression and that they may also display an alternative phenotype corresponding to what are commonly called “cancer stem cells” but in reality are not more undifferentiated cells but are instead more adult-like cells. (see PCT Patent Application Ser. No. PCT/US2020/047707, which is incorporated by reference herein in its entirety). As shown in FIG. 1, the embryonic-like (or De-matured Cancer (DC) cells are generally those with relatively low LMNA, COX7A1, PCDHGA12, and TNFRSF11B expression, and relatively high LMNB1 expression. In the case of carcinomas, these commonly are the epithelial-like cancer cells, are relatively sensitive to radio- and chemotherapy, and relatively non-mobile. In contrast, the Adult-like Cancer (AC) cells are generally those with relatively high LMNA, COX7A1, PCDHGA12, TNFRSF11B, and mesenchymal gene expression (including but not limited to COL1A1, SNAI2, CD44) expression, and relatively high LMNB1 expression. In the case of carcinomas, these commonly are the cancer cells that have undergone epithelial-mesenchymal transformation, are that are relatively insensitive to radio- and chemotherapy, and are relatively prone to metastasis.

### Markers of Pluripotent Stem, Embryonic, Adult, EMT, and CSCs

Gene expression markers that determine the developmental status of cells are useful in research relating to regenerative biology and cancer research. Pluripotent stem cell gene expression markers utilized in the present invention include: TRIM71, DNMT3B, POU5F1 (OCT4), and NANOG. Embryonic-Specific markers include: CPT1B, AMH, LIN28B, and IGF2BP1. Adult markers include: COX7A1, PCDHGA12, MT1E, and XAF1. Epithelial markers include: RBM47, EPCAM, CDS1, and CDH1. Mesenchymal markers include COL1A1, SPARC, VIM, and SNAI2. Cancer stem cell markers include ALCAM, ALDH1A1, CD44, and CD133 (PROM1).

We previously disclosed that certain genes were differentially-expressed in embryonic (pre-fetal) cells of most somatic cell types and other members were expressed in the fetal and adult stages of development in a similarly unexpectedly large number of somatic cell types (see PCT Patent Application Ser. Nos. PCT/US2020/047707 and PCT/US2014/040601, which is incorporated by reference herein in its entirety). We propose herein that lamin A and lamin B play a critical in regulating chromatin architecture in the embryonic stages of development, that LMNB1 is regulated by the transcription factors TCF3 and/or POU2F1, and that the transition to a fetal and adult pattern of expression reflects a post-embryonic inhibition of regenerative potential in that tissue. We furthermore teach that cancers of a similarly diverse and surprisingly large number of cancer types revert to an embryonic pattern of CPL expression, but that said cancer cells are biphasic and can shift between an adult and an embryonic pattern of CPL isoform expression. Furthermore, the present invention teaches that the resulting heterogeneity leads to a spectrum of characteristics within tumors. The embryonic pattern of CPL isoform expression leads to cell-cell aggregation, and is associated with rapid proliferation, increased aerobic glycolysis, and sensitivity to apoptosis such as when exposed to radio- or chemotherapy. The adult pattern of CPL isoform expression leads to a loss of cell-cell aggregation and instead an epithelial-mesenchymal transformation, is associated with slower rates of proliferation, increased oxidative phosphorylation, and relative insensitivity to apoptosis such as when exposed to radio- or chemotherapy. The latter cells are often referred to as cancer cells that have undergone epithelial-mesenchymal transition (EMT) or cancer stem cells (CSCs). Therefore, the present invention teaches the contrary doctrine that CSCs are not more undifferentiated than other cancer cells, but quite the opposite, they are more mature and adult-like. Further, the present invention provides that the transition of the adult status of CPL isoform expression to embryonic expression may occur early in the pathogenesis of cancer. By way of non-limiting example, embryonic isoform expression may occur in intestinal adenomas before the progression to adenocarcinomas occurs. This provides a means of detecting early stages of oncogenesis in all the diverse cell and cancer types disclosed herein as well as means of targeting said cells for therapeutic effect.

In addition, we disclose that the relative high ratio of expression of the lamin B1 (LMNB1) gene compared to lamin A/C (LMNA) organizes chromatin in the CPL leading to hypermethylation of the CPL isoform CGIs, and an embryonic pattern of CPL isoform expression which is characterized by activation of members of the α and β clusters and repression of members of the γ locus with the exception of PCDHGB4 and PCDHGB6 which are relatively highly expressed in embryonic cells. In contrast, the relative high ratio of expression of the gene LMNA compared to LMNB1 organizes chromatin in the CPL leading to decreased methylation of the CPL isoform CGIs, and an adult pattern of CPL isoform expression which is characterized by inhibition of expression of members of the α and β clusters and increased expression of members of the γ locus with the exception of PCDHGB4 and PCDHGB6 which are relatively highly expressed in embryonic cells.

Up-regulation of the α and β CPL isoforms and PCDHGB4 and PCDHGB6 and downregulation of the γ isoforms (with the exception of PCDHGB4 and PCDHGB6) may be achieved by the up-regulation of the LMNB1/LMNA expression ratio. This, in turn, may be effectively accomplished by the exogenously induced expression of LMNB1 with or without the inhibition of expression of LMNA or the inhibition of LMNA with or without the induced expression of LMNB1. This altered gene expression may be accomplished by RNA or DNA-mediated induction of expression or siRNA using the methods described herein.

Therefore these unanticipated results provide novel diagnostic and therapeutic compositions and methods as described below.

### Diagnostic Applications

The identification of altered expression of lamin A (encoded by the gene LMNA) and/or lamin B1 (encoded by the gene LMNB1) in cancer can be utilized in diagnosis and the detection of cancer cells in vivo for targeted removal. In the case of diagnosis, the presence of lamin A (encoded by the gene LMNA) and/or lamin B1 (encoded by the gene LMNB1) expression in a fetus or adult indicates the likelihood of cells progressing towards malignancy or outright malignant cells that are relatively rapidly proliferating and sensitive to radio- or chemotherapy. In contrast, the detection of cancer cells expressing a fetal or adult pattern of lamin A (encoded by the gene LMNA) and/or lamin B1 (encoded by the gene LMNB1) expression identifies cells that have undergone EMT, are relatively resistant to radio- or chemotherapy, and are prone to metastasis. Detection of the embryonic vs fetal-adult state of cells can be accomplished through the detection of transcribed RNA for embryonic lamin A (encoded by the gene LMNA) and/or lamin B1 (encoded by the gene LMNB1) as described herein, or by detecting the lamin A (encoded by the gene LMNA) and/or lamin B1 (encoded by the gene LMNB1) antigens such as through the use of biotin-labeled detection antibodies, or equivalent methods to detect antigens.

Reagents that are capable of detecting lamin A (encoded by the gene LMNA) and/or lamin B1 (encoded by the gene LMNB1) patterns of expression safely in vivo are useful in detecting cancer in real-time wherein a ligand is introduced to the tissue through the circulation, local injection, or topical application wherein said ligand can directly emit light such as with fluorescence allowing a surgeon to precisely demarcate the location of precancerous or cancerous cells for destruction or removal (see e.g., U.S. Pat. No. 9,451,882, which is incorporated by reference herein in its entirety).

### Therapeutic Applications

The teaching of the present invention, in particular, the novel insight that diverse somatic embryonic cells outside of the central nervous system express an embryonic pattern of lamin A (encoded by the gene LMNA) and/or lamin B1 (encoded by the gene LMNB1), and also the insight that cancer cells of diverse cell types have frequently reverted to an embryonic pattern of lamin A (encoded by the gene LMNA) and/or lamin B1 (encoded by the gene LMNB1) expression but said cancer cells can revert to an adult pattern of lamin A (encoded by the gene LMNA) and/or lamin B1 (encoded by the gene LMNB1) expression in what is commonly called epithelial-mesenchymal transition (also inappropriately called “cancer stem cells”) allow numerous therapeutic strategies.

Induced Tissue Regeneration (iTR) and Induced Senolysis of Cancer Stem Cells (iS-CSC) Using CPL Isoforms

We previously disclosed the use of nucleic acids encoded by the genes: OCT4, SOX2, KLF4, NANOG, ESRRB, NR5A2, CEBPA, MYC, TERT, LIN28A and LIN28B (see PCT Patent Application Ser. Nos. PCT/US2017/036452 and PCT/US2014/040601, each of which is incorporated by reference herein in its entirety) for inducing tissue regeneration (iTR) and induced Senolysis of Cancer Stem Cells (iS-CDC), herein we provide improved methods of reprogramming diverse adult cell types to a state that promotes scarless regeneration (iTR) or induced senolysis of cancer stem cells (iS-CSC) and in inducing Tissue Maturation (iTM) by means of targeting lamin A (encoded by the gene LMNA) and/or lamin B1 (encoded by the gene LMNB1).

Critical regions of chromatin such as exemplified by the CPL locus is unexpectedly enclosed in unusually tightly-controlled chromatin reflecting an association with lamin B1 in embryonic cells, and an increased amount of lamin A in fetal and adult cells of various somatic cell types. The region has very high relative levels of H3K9me3 and H4K20me3 histone modification characteristic of heterochromatin such as in peri-centromeric or peri-telomeric DNA. Reprogramming factors, even pioneer factors such as those encoded by the genes SOX2, OCT4, KLF4, and NANOG, therefore inefficiently reprogram embryonic CPL isoform expression in adult cells. Therefore, the improved methods of iTR and/or iS-SCS utilizes two steps that can be performed simultaneously or separated in time, preferably step one occurring first. In the first step, H3K9me3 heterochromatin is relaxed through the inhibition of one or more of the methyltransferases responsible for H3K9me3 methylation; namely, those encoded by the genes SUV39H1, SUV39H2, and SETDB1. Reduction in the activity of these gene products can be readily achieved by methods known in the art such as the use of siRNA targeting SUV39H1 transcript, or preferably SUV39H1, SUV39H2, and SETDB1 transcripts. Alternatively, or in addition small molecule inhibitors of the enzymes can be used such as the SUV39H1 inhibitor Chaetocin, the SUV39H2 inhibitor OTS186935 hydrochloride, and the SETDB1 inhibitors mithramycin A, demycarosyl-3D-β-D-digitoxosyl-mithramycin SK (DIG-MSK), also known as “EC-8042”, streptonigrin and emetine.

In the second step (or less-efficiently as the sole step) the target normal adult or the cancer cells with adult patterns of lamin A (encoded by the gene LMNA) and/or lamin B1 (encoded by the gene LMNB1) expression are transiently exposed to factors that in other conditions are capable of reprogramming the cells to pluripotency. The factors may include constructs that introduce RNA into cells either directly or through gene expression vectors that are capable of inducing pluripotency if allowed to react with cells for a sufficient period of time, but for lesser times can cause iTR. Gene expression vectors are known in the art including, but not limited to, linear polynucleotides, polynucleotides associated with ionic or amphiphilic compounds, plasmids, and viral vectors. Methods of introducing gene expression vectors into a cell are known in the art. Preferably, the RNAs do not include all of the RNAs needed for reprogramming to pluripotency such as only LIN28A or LIN28B optionally together with OCT4 and SOX2, and in the case of inducing tissue regeneration, preferably with an agent to increase telomere length such as RNA for the catalytic component of telomerase (TERT). Most preferably, the agents to induce iTR are genes/factors induced by LIN28A or LIN28B-encoded proteins such as GFER, optionally in combination with an agent that increases telomere length such as the RNA or gene encoding TERT, and/or in combination with the factors disclosed herein important for iTR such as 0.05-5 mM valproic acid, preferably 0.5 mM valproic acid, 1-100 ng/mL AMH, preferably 10 ng/mL AMH, and 2-200 ng/mL GFER, preferably 20 ng/mL. When administered in vivo, such factors are preferably administered in a slow-release hydrogel matrix such as one comprised of chemically modified and crosslinked hyaluronic acid and collagen such as HyStem matrices.

More preferably, factors for iTR or iS-CSC are chosen from agents capable in other conditions of inducing pluripotency in somatic cell types. Such agents include the following compounds individually or in combination: the genes OCT4, SOX2, KLF4, NANOG, ESRRB, NR5A2, CEBPA, MYC, TERT, LIN28A and LIN28B alone and in combination. Nonlimiting examples are the transient expression by AAV vectors transiently expressing from 1-2 weeks the combination of factors LIN28B, OCT4, SOX2, NANOG, and TERT, or alternatively, KLF4, OCT4, SOX2, and TERT at levels comparable to that in normal hES cells. Said factors may also include small molecule compounds such as combinations of the following compounds: inhibitors of glycogen synthase 3 (GSK3) including but not limited to CHIR99021; inhibitors of TGF-beta signaling including but not limited to SB431542, A-83-01, and E616452; HDAC inhibitors including but not limited to aliphatic acid compounds including but not limited to: valproic acid, phenylbutyrate, and n-butyrate; cyclic tetrapeptides including trapoxin B and the depsipeptides; hydroxamic acids such as trichostatin A, vorinostat (SAHA), belinostat (PXD101), LAQ824, panobinostat (LBH589), and the benzamides entinostat (MS-275), CI994, mocetinostat (MGCD0103); those specifically targeting Class I (HDAC1, HDAC2, HDAC3, and HDAC8), IIA (HDAC4, HDAC5, HDAC7, and HDAC9), IIB (HDAC6 and HDAC10), III (SIRT1, SIRT2, SIRT3, SIRT4, SIRT5, SIRT6, or SIRT7) including the sirtuin inhibitors nicotinomide, diverse derivatives of NAD, dihydrocoumarin, naphthopyranone, and 2-hydroxynaphthaldehydes, or IV (HDAC11) deacetylases; inhibitors of H3K4/9 histone demethylase LSD1 including but not limited to parnate; inhibitors of Dot1L including but not limited to EPZ004777; inhibitors of G9a including but not limited to Bix01294; inhibitors of EZH2 including but not limited to DZNep, inhibitors of DNA methyltransferase including but not limited to RG108; 5-aza-2′deoxycytidine (trade name Vidaza and Azadine); vitamin C which can inhibit DNA methylation, increase Tet1 which increases 5hmC which is a first step of demethylation; activators of 3′ phosphoinositide-dependent kinase 1 including but not limited to PS48; promoters of glycolysis including but not limited to Quercetin and fructose 2, 6-bisphosphate (an activator of phosphofructokinase 1); agents that promote the activity of the HIF1 transcription complex including but not limited to Quercetin; RAR agonists including but not limited to AM580, CD437, and TTNPB; agents that mimic hypoxia including but not limited to Resveratrol; agents that increase telomerase activity including but not limited to the exogenous expression of the catalytic component of telomerase (TERT), agents that promote epigenetic modifications via downregulation of LSD1, a H3K4-specific histone demethylase including but not limited to lithium; or inhibitors of the MAPK/ERK pathway including but not limited to PD032590. Such compounds may be administered in diverse combinations, concentrations, and for differing periods of time, to optimize the effect of iTR on cells cultured in vitro using markers of global iTR such as by assaying for decreased expression of COX7A1 or NAALADL1, or other inhibitors of iTR as described herein, and/or assaying for increased expression of PCDHB2 or AMH or other activators or iTR as described herein, or in injured or diseased tissues in vivo, or in modulating the lifespan of animals in vivo.

### Screens, Reporter Molecules, Cells, and Membranes

In general, detectable moieties useful in the reporter molecules of the invention include light-emitting or light-absorbing compounds that generate or quench a detectable fluorescent, chemiluminescent, or bioluminescent signal. In some embodiments, modulation of lamin A (encoded by the gene LMNA) and/or lamin B1 (encoded by the gene LMNB1) causes release of the detectable moiety into a liquid medium, and the signal generated or quenched by the released detectable moiety present in the medium (or a sample thereof) is detected. In some embodiments, the resulting signal causes an alteration in a property of the detectable moiety, and such alteration can be detected, e.g., as an optical signal. For example, the signal may alter the emission or absorption of electromagnetic radiation (e.g., radiation having a wavelength within the infrared, visible or UV portion of the spectrum) by the detectable moiety. In some embodiments, a reporter molecule comprises a fluorescent or luminescent moiety, and a second molecule serves as quencher that quenches the fluorescent or luminescent moiety. Such alteration can be detected using apparatus and methods known in the art.

In many embodiments of the invention, the reporter molecule is a genetically encodable molecule that can be expressed by a cell, and the detectable moiety comprises, e.g., a detectable polypeptide. Thus in some embodiments, the reporter molecule is a polypeptide comprising a fluorescent polypeptides such as green, blue, sapphire, yellow, red, orange, and cyan fluorescent proteins and derivatives thereof (e.g., enhanced GFP); monomeric red fluorescent protein and derivatives such as those known as “mFruits”, e.g., mCherry, mStrawberry, mTomato, etc., and luminescent proteins such as aequorin. (It will be understood that in some embodiments, the fluorescence or luminescence occurs in the presence of one or more additional molecules, e.g., an ion such as a calcium ion and/or a prosthetic group such as coelenterazine.) In some embodiments, the detectable moiety comprises an enzyme that acts on a substrate to produce a fluorescent, luminescent, colored, or otherwise detectable product. Examples of enzymes that may serve as detectable moieties include luciferase; beta-galactosidase; horseradish peroxidase; alkaline phosphatase; etc. (It will be appreciated that the enzyme is detected by detecting the product of the reaction.) In some embodiments, the detectable moiety comprises a polypeptide tag that can be readily detected using a second agent such as a labeled (e.g., fluorescently labeled) antibody. For example, fluorescently labeled antibodies that bind to the HA, Myc, or a variety of other peptide tags are available. Thus the invention encompasses embodiments in which a detectable moiety can be detected directly (i.e., it generates a detectable signal without requiring interaction with a second agent) and embodiments in which a detectable moiety interacts (e.g., binds and/or reacts) with a second agent and such interaction renders the detectable moiety detectable, e.g., by resulting in generation of a detectable signal or because the second agent is directly detectable. In embodiments in which a detectable moiety interacts with a second agent to produce a detectable signal, the detectable moiety may react with the second agent is acted on by a second agent to produce a detectable signal. In many embodiments, the intensity of the signal provides an indication of the amount of detectable moiety present. e.g., in a sample being assessed or in area being imaged. In some embodiments, the amount of detectable moiety is optionally quantified, e.g., on a relative or absolute basis, based on the signal intensity.

The invention provides nucleic acids comprising a sequence that encodes a reporter polypeptide of the invention. In some embodiments, a nucleic acid encodes a precursor polypeptide of a reporter polypeptide of the invention. In some embodiments, the sequence encoding the polypeptide is operably linked to expression control elements (e.g., a promoter or promoter/enhancer sequence) appropriate to direct transcription of mRNA encoding the polypeptide. The invention further provides expression vectors comprising the nucleic acids. Selection of appropriate expression control elements may be based, e.g., on the cell type and species in which the nucleic acid is to be expressed. One of ordinary skill in the art can readily select appropriate expression control elements and/or expression vectors. In some embodiments, expression control element(s) are regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, phosphoglycerate kinase, etc. Regulatable (e.g., inducible or repressible) expression systems such as the Tet-On and Tet-Off systems (regulatable by tetracycline and analogs such as doxycycline) and others that can be regulated by small molecules such as hormones receptor ligands (e.g., steroid receptor ligands, which may or may not be steroids), metal-regulated systems (e.g., metallothionein promoter), etc.

The invention further provides cells and cell lines that comprise such nucleic acids and/or vectors. In some embodiments, the cells are eukaryotic cells, e.g., fungal, plant, or animal cells. In some embodiments, the cell is a vertebrate cell, e.g., a mammalian cell, e.g., a human cell, non-human primate cell, or rodent cell. Often a cell is a member of a cell line, e.g., an established or immortalized cell line that has acquired the ability to proliferate indefinitely in culture (e.g., as a result of mutation or genetic manipulation such as the constitutive expression of the catalytic component of telomerase). Numerous cell lines are known in the art and can be used in the instant invention. Mammalian cell lines include, e.g., HEK-293 (e.g., HEK-293T), CHO, NIH-3T3, COS, and HeLa cell lines. In some embodiments, a cell line is a tumor cell line. In other embodiments, a cell is non-tumorigenic and/or is not derived from a tumor. In some embodiments, the cells are adherent cells. In some embodiments, non-adherent cells are used. In some embodiments, a cell is of a cell type or cell line is used that has been shown to naturally have a subset of iTR reprogramming genes expressed or lamin A (encoded by the gene LMNA) and/or lamin B1 (encoded by the gene LMNB1) or the transcription factors TCF3 or POU2F1 expressed. If a cell lacks one or more said genes, the cell can be genetically engineered to express such protein(s). In some embodiments, a cell line of the invention is descended from a single cell. For example, a population of cells can be transfected with a nucleic acid encoding the reporter polypeptide and a colony derived from a single cell can be selected and expanded in culture. In some embodiments, cells are transiently transfected with an expression vector that encodes the reporter molecule. Cells can be co-transfected with a control plasmid, optionally expressing a different detectable polypeptide, to control for transfection efficiency (e.g., across multiple runs of an assay).

Uses of iTR, iS-CSC, and ICM Factors

### Pharmaceutical Compositions

iTR, iS-CSC, and iCM factors have a variety of different uses. Non-limiting examples of such uses are discussed herein.

In some embodiments, an iTR factor is used to enhance regeneration of an organ or tissue. In some embodiments, an iTR factor is used to enhance regeneration of a limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, an iTR factor is used to enhance regeneration of a stromal layer, e.g., a connective tissue supporting the parenchyma of a tissue. In some embodiments, an iTR factor is used to enhance regeneration following surgery, e.g., surgery that entails removal of at least a portion of a diseased or damaged tissue, organ, or other structure such as a limb, digit, etc. For example, such surgery might remove at least a portion of a liver, lung, kidney, stomach, pancreas, intestine, mammary gland, ovary, testis, bone, limb, digit, muscle, skin, etc. In some embodiments, the surgery is to remove a tumor. In some embodiments, an iTR factor is used to promote scarless regeneration of skin following trauma, surgery, disease, and burns.

Enhancing regeneration can include any one or more of the following, in various embodiments: (a) increasing the rate of regeneration; (b) increasing the extent of regeneration; (c) promoting establishment of appropriate structure (e.g., shape, pattern, tissue architecture, tissue polarity) in a regenerating tissue or organ or other body structure; (d) promoting growth of new tissue in a manner that retains and/or restores function. While use of an iTR factor to enhance regeneration is of particular interest, the invention encompasses use of an iTR factor to enhance repair or wound healing in general, without necessarily producing a detectable enhancement of regeneration. Thus, the invention provides methods of enhancing repair or wound healing, wherein an iTR factor is administered to a subject in need thereof according to any of the methods described herein.

Numerous aspects of aging and age-related disease are taught in the present invention to addressable with iTR therapy. These manifestations of aging include age-related vascular dysfunction including peripheral vascular, coronary, and cerebrovascular disease; musculoskeletal disorders including osteoarthritis, intervertebral disc degeneration, bone fractures, tendon and ligament tears, and limb regeneration; neurological disorders including stroke and spinal cord injuries; muscular disorders including muscular dystrophy, sarcopenia, myocardial infarction, and heart failure; endocrine disorders including Type I diabetes, Addison's disease, hypothyroidism, and pituitary insufficiency; digestive disorders including pancreatic exocrine insufficiency; ocular disorders including macular degeneration, retinitis pigmentosa, and neural retinal degeneration disorders; dermatological conditions including skin burns, lacerations, surgical incisions, alopecia, graying of hair, and skin aging; pulmonary disorders including emphysema and interstitial fibrosis of the lung; auditory disorders including hearing loss; and hematological disorders such as aplastic anemia and failed hematopoietic stem cell grafts.

In some embodiments, the invention provides a method of enhancing regeneration in a subject in need thereof, the method comprising administering an effective amount of an iTR factor to the subject. In some embodiments, an effective amount of a compound (e.g., an iTR factor) is an amount that results in an increased rate or extent of regeneration of damaged tissue as compared with a reference value (e.g., a suitable control value). In some embodiments, the reference value is the expected (e.g., average or typical) rate or extent of regeneration in the absence of the compound (optionally with administration of a placebo). In some embodiments, an effective amount of an iTR factor is an amount that results in an improved structural and/or functional outcome as compared with the expected (e.g., average or typical) structural or functional outcome in the absence of the compound. In some embodiments, an effective amount of a compound, e.g., an iTR factor, results in enhanced blastema formation and/or reduced scarring. Extent or rate of regeneration can be assessed based on dimension(s) or volume of regenerated tissue, for example. Structural and/or functional outcome can be assessed based on, e.g., visual examination (optionally including use of microscopy or imaging techniques such as X-rays, CT scans, MRI scans, PET scans) and/or by evaluating the ability of the tissue, organ, or other body part to perform one or more physiological processes or task(s) normally performed by such tissue, organ, or body part. Typically, an improved structural outcome is one that more closely resembles normal structure (e.g., structure that existed prior to tissue damage or structure as it exists in a normal, healthy individual) as compared with the structural outcome that would be expected (e.g., average or typical outcome) in the absence of treatment with an iTR factor. One of ordinary skill in the art can select an appropriate assay or test for function. In some embodiments, an increase in the rate or extent of regeneration as compared with a control value is statistically significant (e.g., with a p value of <0.05, or with a p value of <0.01) and/or clinically significant. In some embodiments, an improvement in structural and/or functional outcome as compared with a control value is statistically significant and/or clinically significant. “Clinically significant improvement” refers to an improvement that, within the sound judgement of a medical or surgical practitioner, confers a meaningful benefit on a subject (e.g., a benefit sufficient to make the treatment worthwhile). It will be appreciated that in many embodiments an iTR modulator, e.g., an iTR factor, administered to a subject of a particular species (e.g., for therapeutic purposes) is a compound that modulates, e.g., inhibits, the endogenous TR genes expressed in subjects of that species. For example, if a subject is human, a compound that inhibits the activity of human TR inhibitor gene products and activates the activity of human TR activator gene products would typically be administered.

In some embodiments, the iTR factor is used to enhance skin regeneration, e.g., after a burn (thermal or chemical), scrape injury, or other situations involving skin loss, e.g., infections such as necrotizing fasciitis or purpura fulminans. In some embodiments, a burn is a second or third degree burn. In some embodiments a region of skin loss has an area of at least 10 cm². In one aspect, an iTR factor enhances regeneration of grafted skin. In one aspect, an iTR factor reduces excessive and/or pathological wound contraction or scarring.

In some embodiments, an iTR factor is used to enhance bone regeneration, e.g., in a situation such as non-union fracture, implant fixation, periodontal or alveolar ridge augmentation, craniofacial surgery, or other conditions in which generation of new bone is considered appropriate. In some embodiments, an iTR factor is applied to a site where bone regeneration is desired. In some embodiments, an iTR factor is incorporated into or used in combination with a bone graft material. Bone graft materials include a variety of ceramic and proteinaceous materials. Bone graft materials include autologous bone (e.g., bone harvested from the iliac crest, fibula, ribs, etc.), allogeneic bone from cadavers, and xenogeneic bone. Synthetic bone graft materials include a variety of ceramics such as calcium phosphates (e.g. hydroxyapatite and tricalcium phosphate), bioglass, and calcium sulphate, and proteinaceous materials such as demineralized bone matrix (DBM). DBM can be prepared by grinding cortical bone tissues (generally to 100-500 m sieved particle size), then treating the ground tissues with hydrochloric acid (generally 0.5 to 1 N). In some embodiments, an iTR factor is administered to a subject together with one or more bone graft materials. The iTR factor may be combined with the bone graft material (in a composition comprising an iTR factor and a bone graft material) or administered separately, e.g., after placement of the graft. In some embodiments, the invention provides a bone paste comprising an iTR factor. Bone pastes are products that have a suitable consistency and composition such that they can be introduced into bone defects, such as voids, gaps, cavities, cracks etc., and used to patch or fill such defects, or applied to existing bony structures. Bone pastes typically have sufficient malleability to permit them to be manipulated and molded by the user into various shapes. The desired outcome of such treatments is that bone formation will occur to replace the paste, e.g., retaining the shape in which the paste was applied. The bone paste provides a supporting structure for new bone formation and may contain substance(s) that promote bone formation. Bone pastes often contain one or more components that impart a paste or putty-like consistency to the material, e.g., hyaluronic acid, chitosan, starch components such as amylopectin, in addition to one or more of the ceramic or proteinaceous bone graft materials (e.g., DBM, hydroxyapatite) mentioned above.

In some embodiments, an iTR factor enhances the formation and/or recruitment of osteoprogenitor cells from undifferentiated mesechymal cells and/or enhances the differentiation of osteoprogenitor cells into cells that form new bone (osteoblasts).

In some embodiments, an iTR factor is administered to a subject with osteopenia or osteoporosis, e.g., to enhance bone regeneration in the subject.

In some embodiments, an iTR factor is used to enhance regeneration of a joint (e.g., a fibrous, cartilaginous, or synovial joint). In some embodiments, the joint is an intervertebral disc. In some embodiments, a joint is a hip, knee, elbow, or shoulder joint. In some embodiments, an iTR factor is used to enhance regeneration of dental and/or periodontal tissues or structures (e.g., pulp, periodontal ligament, teeth, periodontal bone).

In some embodiments, an iTR factor is used to reduce glial scarring in CNS and PNS injuries.

In some embodiments, an iTR factor is used to reduce adhesions and stricture formation in internal surgery.

In some embodiments, an iTR factor is used to decrease scarring in tendon and ligament repair improving mobility.

In some embodiments, an iTR factor is used to reduce vision loss following eye injury.

In some embodiments, an iTR factor is administered to a subject in combination with cells. The iTR factor and the cells may be administered separately or in the same composition. If administered separately, they may be administered at the same or different locations. The cells can be autologous, allogeneic, or xenogeneic in various embodiments. The cells can comprise progenitor cells or stem cells, e.g., adult stem cells. As used herein, a stem cell is a cell that possesses at least the following properties: (i) self-renewal, i.e., the ability to go through numerous cycles of cell division while still maintaining an undifferentiated state; and (ii) multipotency or multidifferentiative potential, i.e., the ability to generate progeny of several distinct cell types (e.g., many, most, or all of the distinct cell types of a particular tissue or organ). An adult stem cell is a stem cell originating from non-embryonic tissues (e.g., fetal, post-natal, or adult tissues). As used herein, the term “progenitor cell” encompasses cells multipotent and cells that are more differentiated than pluripotent stem cells but not fully differentiated. Such more differentiated cells (which may arise from embryonic progenitor cells) have reduced capacity for self-renewal as compared with embryonic progenitor cells. In some embodiments, an iTR factor is administered in combination with mesenchymal progenitor cells, neural progenitor cells, endothelial progenitor cells, hair follicle progenitor cells, neural crest progenitor cells, mammary stem cells, lung progenitor cells (e.g., bronchioalveolar stem cells), muscle progenitor cells (e.g., satellite cells), adipose-derived progenitor cells, epithelial progenitor cells (e.g., keratinocyte stem cells), and/or hematopoietic progenitor cells (e.g., hematopoietic stem cells). In some embodiments, the cells comprise induced pluripotent stem cells (iPS cells), or cells that have been at least partly differentiated from iPS cells. In some embodiments, the progenitor cells comprise adult stem cells. In some embodiments, at least some of the cells are differentiated cells, e.g., chondrocytes, osteoblasts, keratinocytes, hepatocytes. In some embodiments, the cells comprise myoblasts.

In some embodiments, an iTR factor is administered in a composition (e.g., a solution) comprising one or more compounds that polymerizes or becomes cross-linked or undergoes a phase transition in situ following administration to a subject, typically forming a hydrogel. The composition may comprise monomers, polymers, initiating agents, cross-linking agents, etc. The composition may be applied (e.g., using a syringe) to an area where regeneration is needed, where it forms a gel in situ, from which an iTR factor is released over time. Gelation may be triggered, e.g., by contact with ions in body fluids or by change in temperature or pH, or by light, or by combining reactive precursors (e.g., using a multi-barreled syringe). (See, e.g., U.S. Pat. No. 6,129,761; Yu L, Ding J. Injectable hydrogels as unique biomedical materials. Chem Soc Rev. 37(8):1473-81 (2008)). In some embodiments the hydrogel is a hyaluronic acid or hyaluronic acid and collagen I-containing hydrogel such as HyStem-C described herein. In some embodiments, the composition further comprises cells.

In some embodiments, an iTR factor is administered to a subject in combination with vectors expressing the catalytic component of telomerase. The vector may be administered separately or in the same composition. If administered separately, they may be administered at the same or different locations. The vector may express the telomerase catalytic component from the same species as the treated tissue or from another species. Said co-administration of the iTR factor with the telomerase catalytic component is particularly useful wherein the target tissue is from an aged individual and said individual is from the human species.

Other inventive methods comprise use of an iTR factor in the ex vivo production of living, functional tissues, organs, or cell-containing compositions to repair or replace a tissue or organ lost due to damage. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a functional tissue or organ can be promoted by contacting an iTR factor. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with an iTR factor, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM). Optionally, an iTR factor is administered to the subject prior to, during, and/or following grafting of the ex vivo generated tissue or organ. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with an iTR factor or combinations of factors including those capable of causing a global pattern of iTR gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, an iTR factor or combination of factors is administered directly to or near a site of tissue damage. “Directly to a site of tissue damage” encompasses injecting a compound or composition into a site of tissue damage or spreading, pouring, or otherwise directly contacting the site of tissue damage with the compound or composition. In some embodiments, administration is considered “near a site of tissue damage” if administration occurs within up to about 10 cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, following damage or loss of a tissue, organ, or other structure, an iTR factor is applied to the remaining portion of the tissue, organ, or other structure. In some embodiments, an iTR factor is applied to the end of a severed digit or limb) that remains attached to the body, to enhance regeneration of the portion that has been lost. In some embodiments, the severed portion is reattached surgically, and an iTR factor is applied to either or both faces of the wound. In some embodiments, an iTR factor is administered to enhance engraftment or healing or regeneration of a transplanted organ or portion thereof. In some embodiments, an iTR factor is used to enhance nerve regeneration. For example, an iTR factor may be infused into a severed nerve, e.g., near the proximal and/or distal stump. In some embodiments, an iTR factor is placed within an artificial nerve conduit, a tube composed of biological or synthetic materials within which the nerve ends and intervening gap are enclosed. The factor or factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

iTR, iTM, and iS-CSC factors may be formulated in extracellular vesicles such as exosomes derived from fetal or adult cells can be administered in physiological solutions such as saline, or slow-released in carboxymethyl hyaluronate crosslinked by PEGDA with carboxymethyl-modified gelatin (HyStem-C) to induce therapeutic effect.

In some embodiments, an iTR factor or combinations of factors is used to promote production of hair follicles and/or growth of hair. In some embodiments an iTR factor triggers regeneration of hair follicles from epithelial cells that do not normally form hair. In some embodiments, an iTR factor is used to treat hair loss, hair sparseness, partial or complete baldness in a male or female. In some embodiments, baldness is the state of having no or essentially no hair or lacking hair where it often grows, such as on the top, back, and/or sides of the head. In some embodiments, hair sparseness is the state of having less hair than normal or average or, in some embodiments, less hair than an individual had in the past or, in some embodiments, less hair than an individual considers desirable. In some embodiments, an iTR factor is used to promote growth of eyebrows or eyelashes. In some embodiments, an iTR factor is used to treat androgenic alopecia or “male pattern baldness” (which can affect males and females). In some embodiments, an iTR factor is used to treat alopecia areata, which involves patchy hair loss on the scalp, alopecia totalis, which involves the loss of all head hair, or alopecia universalis, which involves the loss of all hair from the head and the body. In some embodiments, an iTR factor is applied to a site where hair growth is desired, e.g., the scalp or eyebrow region. In some embodiments, an iTR factor is applied to or near the edge of the eyelid, to promote eyelash growth. In some embodiments, an iTR factor is applied in a liquid formulation. In some embodiments an iTR factor is applied in a cream, ointment, paste, or gel. In some embodiments, an iTR factor is used to enhance hair growth after a burn, surgery, chemotherapy, or other event causing loss of hair or hear-bearing skin.

In some embodiments, an iTR factor or combination of factors are administered to tissues afflicted with age-related degenerative changes to regenerate youthful function. Said age-related degenerative changes includes by way of nonlimiting example, age-related macular degeneration, coronary disease, osteoporosis, osteonecrosis, heart failure, emphysema, peripheral artery disease, vocal cord atrophy, hearing loss, Alzheimer's disease, Parkinson's disease, skin ulcers, and other age-related degenerative diseases. In some embodiments, said iTR factors are co-administered with a vector expressing the catalytic component of telomerase to extend cell lifespan.

In some embodiments, an iTR factor or factors are administered to enhance replacement of cells that have been lost or damaged due to insults such as chemotherapy, radiation, or toxins. In some embodiments such cells are stromal cells of solid organs and tissues.

Inventive methods of treatment can include a step of identifying or providing a subject suffering from or at risk of a disease or condition in which in which enhancing regeneration would be of benefit to the subject. In some embodiments, the subject has experienced injury (e.g., physical trauma) or damage to a tissue or organ. In some embodiments the damage is to a limb or digit. In some embodiments, a subject suffers from a disease affecting the cardiovascular, digestive, endocrine, musculoskeletal, gastrointestinal, hepatic, integumentary, nervous, respiratory, or urinary system. In some embodiments, tissue damage is to a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti).

In some embodiments a compound or composition is administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments a compound or composition is administered to a subject at least once within approximately 1-2 weeks, 2-6 weeks, or 6-12 weeks, after a subject has suffered tissue damage and, optionally, at least once thereafter.

In some embodiments of the invention, it may useful to stimulate or facilitate regeneration or de novo development of a missing or hypoplastic tissue, organ, or structure by, for example, removing the skin, removing at least some tissue at a site where regeneration or de novo development is desired, abrading a joint or bone surface where regeneration or de novo development is desired, and/or inflicting another type of wound on a subject. In the case of regeneration after tissue damage, it may be desirable to remove (e.g., by surgical excision or debridement) at least some of the damaged tissue. In some embodiments, an iTR factor is administered at or near the site of such removal or abrasion.

In some embodiments, an iTR factor is used to enhance generation of a tissue or organ in a subject in whom such tissue or organ is at least partially absent as a result of a congenital disorder, e.g., a genetic disease. Many congenital malformations result in hypoplasia or absence of a variety of tissues, organs, or body structures such as limbs or digits. In other instances a developmental disorder resulting in hypoplasia of a tissue, organ, or other body structure becomes evident after birth. In some embodiments, an iTR factor is administered to a subject suffering from hypoplasia or absence of a tissue, organ, or other body structure, in order to stimulate growth or development of such tissue, organ, or other body structure. In some aspects, the invention provides a method of enhancing generation of a tissue, organ, or other body structure in a subject suffering from hypoplasia or congenital absence of such tissue, organ, or other body structure, the method comprising administering an iTR factor to the subject. In some embodiments, an iTR factor is administered to the subject prior to birth, i.e., in utero. The various aspects and embodiments of the invention described herein with respect to regeneration are applicable to such de novo generation of a tissue, organ, or other body structure and are encompassed within the invention.

In some aspects, an iTR factor is used to enhance generation of tissue in any of a variety of situations in which new tissue growth is useful at locations where such tissue did not previously exist. For example, generating bone tissue between joints is frequently useful in the context of fusion of spinal or other joints.

iTR factors may be tested in a variety of animal models of regeneration. In one aspect, a modulator of iTR is tested in murine species. For example, mice can be wounded (e.g., by incision, amputation, transection, or removal of a tissue fragment). An iTR factor is applied to the site of the wound and/or to a removed tissue fragment and its effect on regeneration is assessed. The effect of a modulator of vertebrate TR can be tested in a variety of vertebrate models for tissue or organ regeneration. For example, fin regeneration can be assessed in zebrafish, e.g., as described in (Mathew L K, Unraveling tissue regeneration pathways using chemical genetics. J Biol Chem. 282(48):35202-10 (2007)), and can serve as a model for limb regeneration. Rodent, canine, equine, caprine, fish, amphibian, and other animal models useful for testing the effects of treatment on regeneration of tissues and organs such as heart, lung, limbs, skeletal muscle, bone, etc., are widely available. For example, various animal models for musculoskeletal regeneration are discussed in Tissue Eng Part B Rev. 16(1) (2010). A commonly used animal model for the study of liver regeneration involves surgical removal of a larger portion of the rodent liver. Other models for liver regeneration include acute or chronic liver injury or liver failure caused by toxins such as carbon tetrachloride. In some embodiments, a model for hair regeneration or healing of skin wounds involves excising a patch of skin, e.g., from a mouse. Regeneration of hair follicles, hair growth, re-epithelialization, gland formation, etc., can be assessed.

In some embodiments, an iS-CSC formulation is used to enhance sensitivity of CSCs to apoptosis in response to radio- or chemotherapy. In some embodiments, an iS-CSC formulation is used to enhance sensitivity of carcinomas and adenocarcinomas including but not limited to those of the oro-pharynx, esophagus, stomach, lungs, pancreas, liver, kidney, prostate, breast, urogenital tract; sarcomas including but not limited to chondrosarcomas, osteosarcomas, Ewing's sarcomas, rhrabdomyosarcomas and liposarcomas; neuronal cell tumors including but not limited to gliomas, neuroblastomas, and autonomic nervous system tumors, and blood cell cancers such as leukemias and lymphomas.

In some embodiments, the invention provides a method of enhancing sensitivity of CSCs in a subject in need thereof, the method comprising administering an effective amount of an iS-CSC formulation to the subject. In some embodiments, an effective amount of a compound (e.g., an iS-CSC formulation) is an amount that results in an increased rate or extent of tumor load as compared with a reference value (e.g., a suitable control value). In some embodiments, the reference value is the expected (e.g., average or typical) rate or extent of reduction in tumor mass in the absence of the compound (optionally with administration of a placebo). In some embodiments, an effective amount of an iS-CSC formulation is an amount that results in an improved structural and/or functional outcome as compared with the expected (e.g., average or typical) structural or functional outcome in the absence of the compound. In some embodiments, an effective amount of a compound, e.g., an iS-CSC formulation, results in decreased metastasis. Extent or rate of metastasis can be assessed based on dimension(s) or volume of tumor, for example. Structural and/or functional outcome can be assessed based on, e.g., visual examination (optionally including use of microscopy or imaging techniques such as X-rays, CT scans, MRI scans, PET scans) and/or by evaluating the response of the tumor over time. In some embodiments, an increase in the rate or extent of tumor regression as compared with a control value is statistically significant (e.g., with a p value of <0.05, or with a p value of <0.01) and/or clinically significant. In some embodiments, an improvement in structural and/or functional outcome as compared with a control value is statistically significant and/or clinically significant. “Clinically significant improvement” refers to an improvement that, within the sound judgement of a medical or surgical practitioner, confers a meaningful benefit on a subject (e.g., a benefit sufficient to make the treatment worthwhile). It will be appreciated that in many embodiments an iS-CSC formulation administered to a subject of a particular species (e.g., for therapeutic purposes) is a compound that modulates, e.g., inhibits, the growth and metastasis of the tumor in subjects of that species. For example, if a subject is human, a compound that inhibits the growth and metastasis of the tumor would typically be administered.

In some embodiments, an iS-CSC formulation enhances the sensitivity of carcinomas to radio- or chemotherapy.

In some embodiments, an iS-CSC formulation enhances the sensitivity of adenocarcinomas to radio- or chemotherapy.

In some embodiments, an iS-CSC formulation enhances the sensitivity of sarcomas to radio- or chemotherapy.

In some embodiments, an iS-CSC formulation enhances the sensitivity of mesotheliomas to radio- or chemotherapy.

In some embodiments, an iS-CSC formulation enhances the sensitivity of gliomas to radio- or chemotherapy.

In some embodiments, an iS-CSC formulation enhances the sensitivity of melanomas to radio- or chemotherapy.

In some embodiments, an iS-CSC formulation enhances the sensitivity of neuroblastomas to radio- or chemotherapy.

In some embodiments, an iS-CSC formulation is administered in a composition (e.g., a solution) comprising one or more compounds that polymerizes or becomes cross-linked or undergoes a phase transition in situ following administration to a subject, typically forming a hydrogel. The composition may comprise monomers, polymers, initiating agents, cross-linking agents, etc. The composition may be applied (e.g., using a syringe) to an area where regeneration is needed, where it forms a gel in situ, from which an iS-CSC formulation is released over time. Gelation may be triggered, e.g., by contact with ions in body fluids or by change in temperature or pH, or by light, or by combining reactive precursors (e.g., using a multi-barreled syringe). (See, e.g., U.S. Pat. No. 6,129,761; Yu L, Ding J. Injectable hydrogels as unique biomedical materials. Chem Soc Rev. 37(8):1473-81 (2008)). In some embodiments the hydrogel is a hyaluronic acid or hyaluronic acid and collagen I-containing hydrogel such as HyStem-C described herein. In some embodiments, the composition further comprises cells.

In some embodiments, an iS-CSC formulation is administered to a subject in combination with an inhibitor of the catalytic component of telomerase. The inhibitor may be administered separately or in the same composition. If administered separately, they may be administered at the same or different locations. The inhibitor may inhibit the activity of the telomerase catalytic component from the same species as the treated tissue or from another species.

In some embodiments, a matrix or scaffold coated or impregnated with an iS-CSC formulation or combinations of factors is implanted, optionally in combination with immune cells targeting the tumor, such as those that recognize the embryonic CPL isoform into a subject in need of regeneration.

In some embodiments, an iS-CSC formulation or combination of factors is administered directly to or near a site of a tumor. “Directly to a site of a tumor” encompasses injecting a compound or composition into a site of tumor or spreading, pouring, or otherwise directly contacting the site of the tumor with the compound or composition. In some embodiments, administration is considered “near a site of the tumor” if administration occurs within up to about 10 cm away from a visible or otherwise evident edge of a site of the tumor or to a blood vessel (e.g., an artery) that is located at least in part within the tumor. Administration “near a site of a tumor” is sometimes administration within an afflicted organ, but at a location where tumor is not evident. In some embodiments, following resection or reduction in tumor mass, an iS-CSC formulation is applied to the remaining portion of the tissue, organ, or other structure. The factor or factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

iTR, iTM, and iS-CSC factors may be formulated in extracellular vesicles such as exosomes derived from fetal or adult cells can be administered in physiological solutions such as saline, or slow-released in carboxymethyl hyaluronate crosslinked by PEGDA with carboxymethyl-modified gelatin (HyStem-C) to induce therapeutic effect.

In some applications, an iS-CSC formulation is used to increase sensitivity of precancerous as well as cancerous cells of the digestive, endocrine, musculoskeletal, gastrointestinal, hepatic, integumentary, nervous, respiratory, or urinary system. In some embodiments, heperplasia is in a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti).

In some embodiments an iS-CSC formulation is administered to a subject. For a period of 1-2 weeks. In some embodiments a compound or composition is re-administered to a subject at least once within approximately 1-2 weeks, 2-6 weeks, or 6-12 weeks, after a subject has received a first treatment.

The compounds and compositions disclosed herein and/or identified using a method and/or assay system described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. Suitable preparations, e.g., substantially pure preparations, of one or more compound(s) may be combined with one or more pharmaceutically acceptable carriers or excipients, etc., to produce an appropriate pharmaceutical composition suitable for administration to a subject. Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For oral administration, compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP).

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection. Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 mg/kg to about 100 mg/kg body weight, about 0.01 to about 25 mg/kg body weight, about 0.1 to about 20 mg/kg body weight, about 1 to about 10 mg/kg. Other exemplary doses include, for example, about 1 μg/kg to about 500 mg/kg, about 100 μg/kg to about 5 mg/kg. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc. Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more compounds or compositions are administered in combination, e.g., for purposes of enhancing regeneration. Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more iTR factors, or vectors expressing the catalytic component of telomerase and an iTR factor, are administered. In some embodiments an iTR factor is administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any of FGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Certain of the inventive methods are often practiced using populations of cells, e.g., in vitro or in vivo. Thus references to “a cell” should be understood as including embodiments in which the cell is a member of a population of cells, e.g., a population comprising or consisting of cells that are substantially genetically identical. However, the invention encompasses embodiments in which inventive methods is/are applied to an individual cell. Thus, references to “cells” should be understood as including embodiments applicable to individual cells within a population of cells and embodiments applicable to individual isolated cells.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. It is contemplated that all embodiments described herein are applicable to all different aspects of the invention. It is also contemplated that any of the embodiments can be freely combined with one or more other such embodiments whenever appropriate. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. Furthermore, where the claims recite a composition, the invention provides methods of making the composition, e.g., according to methods disclosed herein, and methods of using the composition, e.g., for purposes disclosed herein. Where the claims recite a method, the invention provides compositions suitable for performing the method, and methods of making the composition. Also, where the claims recite a method of making a composition, the invention provides compositions made according to the inventive methods and methods of using the composition, unless otherwise indicated or unless one of ordinary skill in the art would recognize that a contradiction or inconsistency would arise. Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value). A “composition” as used herein, can include one or more than one component unless otherwise indicated. For example, a “composition comprising an activator or a TR activator” can consist or consist essentially of an activator of a TR activator or can contain one or more additional components. It should be understood that, unless otherwise indicated, an inhibitor or a TR inhibitor (or other compound referred to herein) in any embodiment of the invention may be used or administered in a composition that comprises one or more additional components including the presence of an activator of a TR activator.

### Methods

In addition to the methods described below, methods that find use in the production and use of cells with an embryonic pattern of gene expression corresponding with scarless regenerative potential can be found in the following: PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, (See, e.g. U.S. provisional patent application No. 61/831,421, filed Jun. 5, 2013, PCT patent application PCT/US2014/040601, filed Jun. 3, 2014 and U.S. patent application Ser. No. 14/896,664, filed on Dec. 7, 2015, the disclosures of which are incorporated by reference in their entirety), each of which is incorporated by reference herein in its entirety.

Induced Senolysis of Cancer Stem Cells (iS-CSC)

In addition, or in contrast, in cases where malignant cells have reverted to a post-EFT phenotype (AC cells) (surprisingly also known as what are commonly designated cancer stem cells), thereby becoming relatively resistant to apoptosis, the resistant “cancer stem cells” can be induced back to a pre-fetal phenotype to increase their susceptibility to treatments that induce apoptosis. These include the reprogramming of said AC cells using iTR reprogramming methods disclosed herein and previously disclosed in PCT Patent Application Ser. Nos. PCT/US2014/040601, PCT/US2017/036452, PCT/US2020/025512 and PCT/US2019/028816, each of which are incorporated in its entirety, (also known as the induction of senolysis of cancer stem cells (iS-CSC)), inhibiting the PI3K/AK T/mTOR (phosphoinositide 3-kinase/AKT/mammalian target of rapamycin) pathway such as with rapamycin or other inhibitors of mTOR, dietary restriction, or the use of dietary restriction mimetics. These and related uses of pathways related to the EFT in the diagnosis and treatment of cancer are the subject of the present invention.

### Novel Cancer Therapeutic Strategies

The methods and compositions of the present invention also provide for novel cancer therapeutics and companion diagnostics. The present invention teaches that certain molecular pathways associated with the EFT evolved in part as a method to restrain the replication of endogenous transposable elements and viruses including Class I transposable elements (retrotransposons), Class II transposable elements (DNA transposons), LINES, SINES, as well as other viruses such as retroviruses. Prior to the EFT and in mammalian pre-implantation embryos, some cells, such as cells of the inner cell mass or cells isolated from the inner cell mass such as cultured hES cells, are permissive for viral replication. The relative permissivity of some embryonic (pre-fetal) cells to endogenous transposable element replication is known in the art. For example, it is documented that human endogenous retroviruses such as HERVK replicate in some pluripotent stem cell lines (Grow, E. J. et al, (2015) Nature 522:221-225). However, the association of Lamin-A with the EFT and the suppression of viral replication has not been described.

The present invention teaches that lamin-A, in particular, its processing into mature filaments and association with LRRK2 and PLPP7 evolved as a means of guarding the integrity of the genome, in particular, regions of repetitive sequences such as those associated with telomeric repeats and tandemly-repeated paralogs such as those of the clustered protocadherin locus or regions of tandemly-repeated paralogs of zinc finger proteins that evolved to inactivate diverse viral sequences. In addition, Lamin A evolved as a means of limiting the plasticity of diverse differentiated somatic types, that is, stabilizing them in their differentiated state. In limiting their plasticity, it limited the potential of diverse somatic cell types and tissues to regenerate after injury or disease by utilizing diverse pathways. These pathways included the downregulation of the embryonic cell-cell recognition system of the clustered protocadherin locus (See, e.g. U.S. provisional patent application No. 63/155,631, filed Mar. 2, 2021, the disclosure of which is incorporated by reference in its entirety) and increased signaling associated with the epithelial-mesenchymal transformation (EMT) such as increased expression of extracellular matrix proteins such as those encoded by the genes: FN1, COL1A1, SPARC, and VIM that result in a fibrotic scarring of adult tissue in lieu of regeneration as seen in embryonic tissue following injury. As a result, Lamin A plays an important regulatory role as an inhibitor of tissue regeneration, but also the formation of cancer stem cells (CSC) which have been disclosed to be not a more undifferentiated cell type as is the current consensus belief, but rather a more mature cell type corresponding to fetal/adult cells, as opposed to the embryonic (pre-fetal) state of many malignant cell types from diverse somatic cell origins.

In another aspect, genes regulating lamin A and/or lamin B1 expression are altered such that cancer cells are treated with agents that alter the expression of the genes from that of an embryonic state to that of a fetal or adult state to cause iCM.

In another aspect, genes regulating lamin A and/or lamin B1 expression are altered such that cancer cells are treated with agents that alter the expression of the genes from that of a fetal or adult state to that of an embryonic state to cause iS-CSC.

### Oncolytic Viral Therapy

The permissive state of pre-EFT somatic cells therefore is consistent with the permissive replication of diverse viruses in cancer cells. While there are currently no efficient means of determining in advance which tumors or cancer cells types will be efficiently destroyed by said vectors, the embryonic and adult gene expression markers in previously disclosures [See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, PCT patent application PCT/US2017/036452, filed Jun. 7, 2017 and U.S. patent application Ser. No. 16/211,690, filed on Dec. 6, 2018, and U.S. provisional patent application No. 63/256,286, filed Oct. 15, 2021, the disclosures of which are incorporated by reference in their entirety], as well as the differentially-methylated DNA sequences associated with embryonic vs fetal/adult cells (see, e.g. PCT patent application PCT/US2020/047707, filed Aug. 25, 2020, the disclosure of which is incorporated by reference in its entirety), provide useful means of determining which cancer cells or tumors will respond to oncolytic viral therapy. Cancer cells or tumors that express embryonic (pre-fetal) markers such as a lack of COX7A1 expression, relatively low expression of LMNA, or alternatively express embryonic (pre-fetal) markers such as the expression of PCAT7, are permissive for the replication of viruses and are therefore sensitive to oncolytic viral therapy. In addition, methods of inducing tissue regeneration such as those disclosed in [(See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, e.g. PCT patent application PCT/US2017/036452, filed Jun. 7, 2017 and U.S. patent application Ser. No. 16/211,690, filed on Dec. 6, 2018, and U.S. provisional patent application No. 63/256,286, filed Oct. 15, 2021, the disclosures of which are incorporated by reference in their entirety) are useful in transforming CSCs into their embryonic counterparts wherein the cancer cells will be responsive to oncolytic viral therapy.

The novel oncolytic viral therapies of the present invention include the use of viruses currently-disclosed as selectively destroying malignant cancer cells including: Herpes Simplex Virus Type I (HSV-1) such as Talimogene laherparepvec (T-VEC) modified to express GM-CSF with a promoter of an embryonic (pre-fetal) gene promoter such as the PCAT7, CPT1B, or PURPL promoters or other embryonic promoters previously disclosed (See, e.g. U.S. provisional patent application No. 63/256,284, filed Oct. 15, 2021, the disclosure of which is incorporated by reference in its entirety).

In addition, viruses useful in targeting cancer cells such as HSV-1, reovirus, picornaviruses (coxsackeievirus, rigavirus) rhabdoviruses such as vesicular stomatitis virus and Maraba virus, and paramyxoviruses such as Newcastle disease virus and Measles virus, and vaccinia virus may be modified to express toxic gene products or genes useful to express specifically in cancer cells such as GM-CSF that are useful in promoting dendritic cell activation wherein said introduced genes are expressed from a gene promoter such as the PCAT7, CPT1B, or PURPL promoters or other embryonic promoters previously disclosed (See, e.g. U.S. provisional patent application No. 63/256,284, filed Oct. 15, 2021, the disclosure of which is incorporated by reference in its entirety).

In addition, viruses useful in targeting cancer cells such as HSV-1, reovirus, picornaviruses (coxsackeievirus, rigavirus) rhabdoviruses such as vesicular stomatitis virus and Maraba virus, and paramyxoviruses such as Newcastle disease virus and Measles virus, and vaccinia virus may be modified to express RNAi to zinc finger protein genes that are activated in fetal/adult cells wherein said zinc finger proteins inhibit viral replication. As a result, infected cells, such as cancer cells with an fetal/adult-like phenotype are rendered more susceptible to lysis. Said fetal/adult-onset zinc finger genes activated by Lamin A include: ZNF280D (See, e.g. U.S. provisional patent application No. 61/831,421, filed Jun. 5, 2013, PCT patent application PCT/US2014/040601, filed Jun. 3, 2014 and U.S. Pat. No. 10,961,531, filed on Dec. 7, 2015, the disclosures of which are incorporated by reference in their entirety), ZNF300P1, ZNF-572 (See, e.g. PCT patent application PCT/US2017/036452, filed Jun. 7, 2017 and U.S. patent application Ser. No. 16/211,690, filed on Dec. 6, 2018, the disclosures of which are incorporated by reference in their entirety), and ZNF578, ZNF585B, ZNF736, and ZNF790-AS1 (See, e.g. U.S. provisional patent application No. 63/256,286, filed Oct. 15, 2021, the disclosure of which is incorporated by reference in its entirety).

In addition, the present invention provides for novel oncolytic viral therapy which when used alone or in combination with immune checkpoint inhibition, or adoptive immunotherapy, are useful in selectively destroying cancer cells with an embryonic phenotype. Numerous immune checkpoint inhibitors useful in treating cancer are known in the art and may be utilized as a combination therapy with the cancer therapeutics described herein. Nonlimiting examples of immune checkpoint inhibitors antibodies targeting PD-1 such as Nivolumab, Cemiplimab, Spartalizumab, and Pembrolizumab and antibodies targeting PD-L1 such as Atezolizumab, Avelumab, and Durvalumab, and antibodies targeting CTLA4 such as Ipilimumab. Additional immune checkpoint inhibition can be achieved by T-Cell Adoptive Cancer Immunotherapy. Said T-Cells are used wherein they express decreased levels of or have a knock-out of CISH (cytokine-inducible SH2-containing protein) or CBLB (Cbl Proto-oncogene, E3 Ubiquitin Protein Ligase B).

Additional combinations that are useful in achieving greater levels of reduction in tumor burden can be achieved by combining the oncolytic viruses of the present invention with the above mentioned immune checkpoint inhibitors, together with dendritic cell therapy and/or CAR-T cells targeting embryonic (pre-fetal) antigens such as those described in (See, e.g. U.S. provisional patent application No. 63/155,631, filed Mar. 2, 2021, the disclosure of which is incorporated by reference in its entirety).

The phenotypic alterations of the EFT are shared in common with the majority of all somatic cell types. Similarly, the abnormal embryonic phenotype (embryo-onco phenotype) of many cancer cells and the fetal/adult phenotype of CSCs are shared by many cancer types (i.e. are pan-cancer phenotypic alterations). They are useful in the diagnosis of primary and metastatic cancers including: Acanthoma, Acinar adenocarcinoma, Acinic cell carcinoma, Acrospiroma, Acute eosinophilic leukemia, Acute erythroid leukemia, Acute Lymphoblastic Leukemia (ALL), Acute megakaryoblastic leukemia, Acute monocytic leukemia, Acute Myeloid Leukemia (AML), Acute promyelocytic leukemia, Adamantinoma, Adenoid cystic carcinoma, Adenomatoid odontogenic tumor, Adenosquamous carcinoma, Adenosquamous lung carcinoma, Adipose tissue neoplasm, Adrenocortical carcinoma, Adrenocortical carcinoma childhood, Aggressive NK-cell leukemia, AIDS-related cancers, Alveolar rhabdomyosarcoma, Alveolar soft part sarcoma, Ameloblastic fibroma, Anal cancer, Anaplastic carcinoma, Anaplastic large-cell lymphoma, Anaplastic thyroid cancer, Angioimmunoblastic T-cell lymphoma, Angiosarcoma, Appendix cancer, Attenuated familial adenomatous polyposis, Atypical teratoid/rhabdoid tumor central nervous system childhood, B-cell chronic lymphocytic leukemia, B-cell lymphoma, Bellini duct carcinoma, Bile duct cancer, Bile duct cancer—Cholangiocarcinoma, Bladder cancer, Bladder cancer—Small cell carcinoma, Bladder cancer—Transitional cell carcinoma, Bladder cancer childhood, Blastoma, Bone cancer, Bone cancer—Osteosarcoma, Brain stem glioma, Brain tumors—other, Brain tumor—Glioblastoma multiforme, Brain tumor—Oligodendroglioma anaplastic, Brain tumor—cerebellar astrocytoma (childhood & adult), Brain tumor—cerebral astrocytoma/malignant glioma (childhood & adult), Brain tumor—ependymoma, Brain tumor—medulloblastoma, Brain tumor—supratentorial primitive neuroectodermal tumors, Brain tumor—visual pathway and hypothalamic glioma, Brain and spinal cord tumors childhood, Breast cancer, Breast cancer ductal adenocarcinoma, Breast cancer childhood, Brenner tumour, Bronchial adenomas/carcinoids, Bronchial tumors, Bronchial tumors childhood, Bronchioloalveolar carcinoma, Brown tumor, Burkitt lymphoma, Carcinoid tumor, Carcinoid tumor childhood, Carcinoid tumor gastrointestinal, Carcinoma of the penis, Carcinosarcoma, Cementoma, Central nervous system cancer, Cervical cancer—adenocarcinoma, Cervical cancer—squamous cell, Cervical Cancer—Neuroendocrine, Carcinoma of the cervix, Cervical cancer childhood, Childhood cancers, Childhood leukemia, Cholangiocarcinoma, Cholangiosarcoma, Chondromyxoid fibroma, Chondrosarcoma, Chordoma, Chorioadenoma destruens, Chorioblastoma, Choriocarcinoma, Choroid plexus tumor, Chorioepithelioma, Clear cell adenocarcinoma, Clear cell adenocarcinoma of the vagina, Clear-cell ovarian carcinoma, Clear-cell sarcoma of the kidney, Colon cancer, Colon cancer—adenocarcinoma, Colorectal cancer, Colorectal cancer childhood, Comedocarcinoma, Craniopharyngioma, Craniopharyngioma childhood, Cutaneous lymphoma, Cystadenocarcinoma, Degos disease, Dermatofibrosarcoma protuberans, Desmoplastic small round cell tumor, Diffuse large B-cell lymphoma, Digestive system neoplasm, Diktyoma, Ductal carcinoma In situ (DCIS), “Ductal, lobular, and medullary neoplasms”, Duodenal cancer, Dysembryoplastic neuroepithelial tumour, Dysgerminoma, ELM4-ALK positive lung cancer, Embryoma, Embryonal carcinoma, Embryonal rhabdomyosarcoma, Embryonal tumors central nervous system childhood, Endocrine gland neoplasm, Endodermal sinus tumor, Endometrial cancer, Endometrial—Stromal sarcoma, Endometrial—Adenocarcinoma, Endometrioid tumor, Enteropathy-associated T-cell lymphoma, Ependymoblastoma childhood, Ependymoma childhood, Epithelial-myoepithelial carcinoma of the lung, Epithelioid sarcoma, Epithelioma, Esophageal cancer, Esophageal cancer childhood, Esthesioneuroblastoma childhood, Ewing family of tumors, Ewing's sarcoma in the Ewing family of tumors, Exocrine cancer, Extracranial germ cell tumor childhood, Extragonadal germ cell tumor, Extrahepatic bile duct cancer, Extramammary Paget's disease, Eye cancer, “Eye cancer, intraocular melanoma”, “Eye cancer, retinoblastoma”, Fallopian tube cancer, Familial adenomatous polyposis, Fetal adenocarcinoma, Fibroepithelial neoplasms, Fibrolamellar hepatocellular carcinoma, Fibrosarcoma, Fibrous tissue neoplasm, Follicular lymphoma, Follicular thyroid cancer, GCB Diffuse Large B-Cell Lymphoma (DLBCL), Gallbladder cancer, Ganglioglioma, Ganglioneuroma, Gardner's syndrome, Gastric carcinoid, Gastric (stomach) cancer, Gastric (stomach) cancer—Adenocarcinoma, Gastric (stomach) cancer—Adenocarcinoma of gastroesophageal junction, Gastric (stomach) cancer childhood, Gastric lymphoma, Gastrinoma, Gastrointestinal carcinoid tumor, Gastrointestinal stromal tumors (GIST), Germ cell tumor, Extragonadal germ cell tumor, Ovarian germ cell tumor, Germinoma, Gestational choriocarcinoma, Gestational trophoblastic tumor, Giant-cell fibroblastoma, Giant-cell glioblastoma, Giant-cell tumor of bone, Gigantiform cementoma, Glial tumor, Gliomatosis cerebri, Glioblastoma Multiforme, Glioma, Glioma childhood visual pathway and hypothalamic, Gliosarcoma, Glucagonoma, Goblet cell carcinoid, Gonadoblastoma, Granulosa cell tumour, Gynandroblastoma, Head and neck cancer, Head and neck cancer childhood, Heart cancer, Hemangioblastoma, Hemangiopericytoma, Hemangiosarcoma, Hematological malignancy, Hepatic cancer—Cholangiocarcinoma, Hepatoblastoma, Hepatocellular (liver) cancer, Hepatosplenic T-cell lymphoma, Hereditary breast-ovarian cancer syndrome, Hereditary nonpolyposis colorectal cancer, Histiocytic sarcoma, Histiocytoma, Hypopharyngeal cancer, Inflammatory breast cancer, Inflammatory myeloblastic tumor, Intraductal carcinoma, Intraductal papillary mucinous neoplasm, Intraocular melanoma, Intratubular germ cell neoplasia, Invasive lobular carcinoma, Islet cell carcinoma, Islet cell tumors (endocrine pancreas), Juvenile granulosa cell tumor, Juvenile myelomonocytic leukemia, Juxtaglomerular cell tumor, Kaposi sarcoma, Kidney cancer childhood, Klatskin tumor, Krukenberg tumor, Langerhans cell histiocytosis, Large-cell lung carcinoma with rhabdoid phenotype, Laryngeal cancer, Laryngeal cancer—squamous cell carcinoma, Laryngeal cancer childhood, Leiomyosarcoma, Lentigo malignant melanoma, Leptomeningeal cancer, Leukemias, Leydig cell tumour, Chronic lymphocytic leukemia (CLL), Chronic myelogenous leukemia (CML), Hairy cell leukemia, Linitis plastica, Lip and oral cavity cancer, Liposarcoma, Liver cancer (primary), Lobular carcinoma, Lobular carcinoma In situ (LCIS), Giant-cell carcinoma of the lung, Large-cell lung carcinoma, Large-cell lung carcinoma with rhabdoid phenotype, Non-small cell lung cancer, Lung—Adenocarcinoma, Lung—Large cell_carcinoma, Lung—Small cell_carcinoma, Lung—Squamous cell_carcinoma, Luteoma, Lymphangioma, Lymphangiosarcoma, Lymphoepithelioma, Lymphomas, Lymphoma—Extranodal marginal zone B-cell of lymphoid tissue, Lymphoma—Follicular cancer of lymphoid tissue, AIDS-related_lymphoma, Cutaneous T-cell lymphoma, Hodgkin_lymphoma, Non-hodgkin lymphoma, Primary central nervous system lymphoma (CNS), Macroglobulinemia Waldenstrim, Male breast cancer, Malignant fibrous histiocytoma of bone and osteosarcoma, Malignant peripheral nerve sheath tumor, Malignant triton tumor, MALT lymphoma, Mammary ductal carcinoma, Mantle cell lymphoma, Marginal zone B-cell lymphoma, “Marcus Whittle, deadly disease”, Mast cell leukemia, Mediastinal germ cell tumor, Mediastinal tumor, Medullary carcinoma, Medullary carcinoma of the breast, Medullary thyroid cancer, Medulloblastoma, Medulloblastoma childhood, Medulloepithelioma, Medulloepithelioma childhood, Melanoma, Melanoma childhood, Meningioma, Merkel cell carcinoma, Mesenchymal chondrosarcoma, Mesothelioma adult malignant, Mesothelioma adult malignant—pleural mixed, Mesothelioma childhood, Metastatic breast cancer, Metastatic squamous neck cancer with occult primary, Metastatic tumor of jaws, Metastatic urothelial carcinoma, Mixed Mullerian tumor, Mouth cancer, Mucinous cystadenocarcinoma of the lung, Mucinous tumor, Multiple endocrine neoplasia syndromes childhood, Multiple endocrine neoplasia type 2b, Multiple myeloma/plasma cell neoplasm, Muscle tissue neoplasm, Mycosis fungoides, Myelodysplastic/myeloproliferative neoplasms, Myelodysplastic syndromes, Myeloid leukemia adult acute, Myeloid leukemia childhood acute, Myeloid sarcoma, Chronic myeloproliferative disorders, Myosarcoma, Myxoid chondrosarcoma, Myxoid liposarcoma, Myxoma, Myxosarcoma, Nasal cavity and paranasal sinus cancer, Nasopharyngeal angiofibroma, Nasopharyngeal cancer, Nasopharyngeal cancer childhood, Nerve sheath tumor, Nervous system neoplasm, Neuroblastoma, Neurocytoma, Neurofibroma, Neuroma, Nipple adenoma, Nodular lymphocyte predominant Hodgkin's lymphoma, Nodular melanoma, Odontogenic tumor, Oncocytoma, Optic nerve sheath meningioma, Optic nerve tumor, Oral cancer, Oral cancer childhood, Oropharyngeal cancer, Oropharyngeal squamous cell carcinomas, Osteolipochondroma, Osteoma, Osteosarcoma, Ovarian cancer, Ovarian cancer—Adenocarcinoma of ovary serous, Ovarian cancer childhood, Ovarian cancer epithelial, Ovarian cancer germ cell tumor, Paget's disease of the breast, Pancoast tumor, Pancreatic cancer, Pancreatic cancer childhood, Pancreatic cancer—Neuroendocrine, Pancreatic cancer islet cell tumors, Pancreatic—Adenocarcinoma of pancreas ductal, Pancreatic serous cystadenoma, Papillary adenocarcinoma, Papillary serous cystadenocarcinoma, Papillary thyroid cancer, Papillomatosis childhood, Paraganglioma, Parathyroid adenoma, Parathyroid cancer, Parathyroid neoplasm, PEComa, Periampullary cancer, Peritoneal mesothelioma, Pharyngeal Cancer, Pheochromocytoma, Pineal astrocytoma, Pineal germinoma, Pineal parenchymal tumors of intermediate differentiation childhood, Pinealoblastoma, Pineoblastoma and supratentorial primitive neuroectodermal tumors childhood, Pineocytoma, Pituicytoma, Pituitary adenoma, Pituitary tumor, Plasma cell dyscrasia, Plasma cell leukemia, Plasma cell neoplasm/multiple myeloma, Plasmacytoma, Pleomorphic undifferentiated sarcoma, Pleomorphic xanthoastrocytoma, Pleuropulmonary blastoma, Pleuropulmonary blastoma childhood, Polyembryoma, Posterior urethral cancer, Precursor T-lymphoblastic lymphoma, Primary peritoneal carcinoma, Primitive neuroectodermal tumor, Prostate cancer, Prostate cancer—adenocarcinoma, Pseudomyxoma peritonei, Rectal cancer, Rectal cancer—adenocarcinoma, Renal cell carcinoma (kidney cancer), Renal medullary carcinoma, Renal pelvis and ureter transitional cell cancer, Reninoma, Respiratory tract neoplasm, Retinoblastoma, Rhabdomycin, Rhabdomyosarcoma childhood, Richter's transformation, Salivary gland cancer, Salivary gland cancer childhood, Salivary gland-like carcinoma of the lung, Salivary gland neoplasm, Sacrococcygeal teratoma, Sarcoma, Sarcoma botryoides, Sarcoma soft tissue, Sarcomatoid carcinoma, Schwannomatosis, Sclerosing rhabdomyosarcoma, Secondary neoplasm, Seminoma, Serous carcinoma, Serous cystadenocarcinoma, Serous tumour, Sertoli cell tumour, Sertoli-Leydig cell tumour, Sex cord-gonadal stromal tumour, Sçzary syndrome, Signet ring cell carcinoma, Skin cancer, Skin cancer childhood, Skin cancer—basal cell carcinoma, Skin cancer—basal-like carcinoma, Skin cancer—melanoma, Small-cell carcinoma, Small intestine cancer, “Small-, round-, blue-cell tumour”, Somatostatinoma, Soot wart, Spermatocytic seminoma, Spinal tumor, Spindle cell cancer, Spindle cell rhabdomyosarcoma, Splenic lymphoma with villous lymphocytes, Splenic marginal zone lymphoma, Squamous cell carcinoma, Squamous neck cancer with occult primary metastatic, Stewart_Treves syndrome, Stromal tumor, Supratentorial primitive neuroectodermal tumors childhood, Surface epithelial-stromal tumor, Synovial sarcoma, T-cell lymphoma, T-lymphoblastic lymphoma, Teratocarcinoma, Testicular cancer, Testicular cancer—Seminoma, Testicular cancer childhood, Thecoma, Throat cancer, “Thymoma, childhood”, Thymoma and thymic carcinoma, Thymoma and thymic carcinoma childhood, Thyroid cancer, Thyroid cancer—follicular, Thyroid cancer—papillary, Thyroid cancer childhood, Tonsil—Carcinoma of tonsil squamous cell, Trabecular cancer, Tracheal tumor, Transitional cell carcinoma, Trophoblastic tumor gestational, Tubulovillous adenoma, Urachal cancer, Ureteral cancer, Ureteral neoplasm, Urethral cancer, Urogenital neoplasm, Urothelial carcinoma, Urothelial cell carcinoma, Uterine cancer, Uterine cancer endometrial, Uterine clear cell carcinoma, Uterine sarcoma, Uterine serous carcinoma, Uveal melanoma, Vaginal cancer, Vaginal cancer childhood, Verrucous carcinoma, Vestibular schwannoma, VIPoma, Visual pathway glioma, Von Hippel_Lindau disease, Vulvar Cancer, “Wilms tumor (kidney cancer), childhood”.

### EXAMPLES

Certain conventional techniques of cell biology, cell culture, molecular biology, microbiology, recombinant nucleic acid (e.g., DNA) technology, immunology, etc., which are within the skill of the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3.sup.rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005, Monoclonal antibodies: a practical approach (P. Shepherd and C Dean, eds., Oxford University Press, 2000); Freshney, R. I., “Culture of Animal Cells, A Manual of Basic Technique”, 5th ed., John Wiley & Sons, Hoboken, N J, 2005). All patents, patent applications, websites, databases, scientific articles, and other publications mentioned herein are incorporated herein by reference in their entirety.

### Example 1: LMNA and LMNB1 Expression are Inversely Correlated During Development

RNA-sequencing was performed on human cells spanning development and aging. In the case of pluripotent stem cells representing one of the earliest stages of development, cells included hES cells and hiPS cells. To standardize somatic cells, dermal fibroblasts at diverse gestational (8-16 weeks gestational age) and post-natal ages (22-83 years of age) were used.

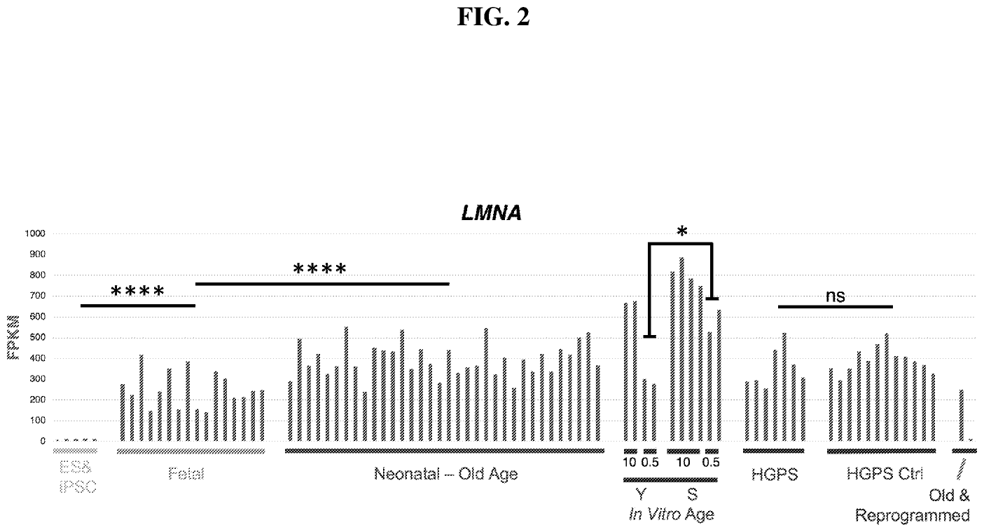

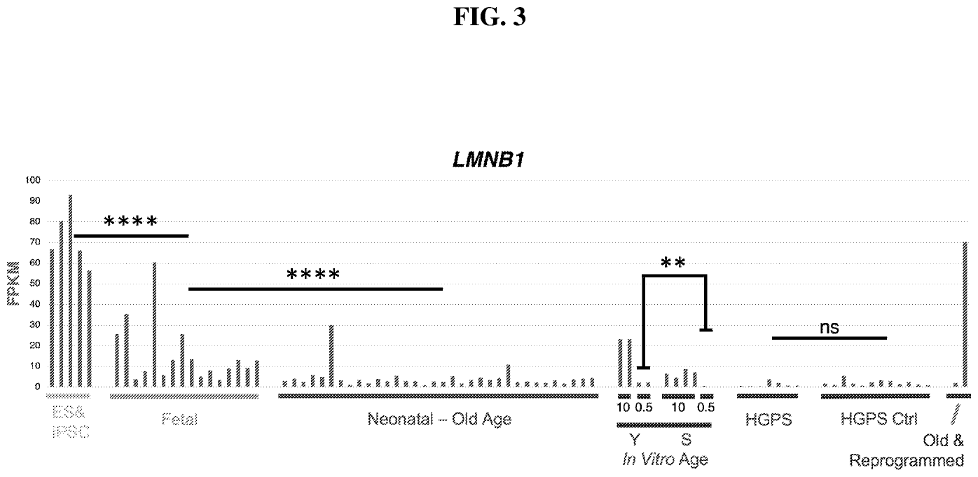

All were sourced from the medial aspect of the upper arm and all were synchronized in quiescence by placing the cells in medium supplemented in 0.5% FBS for five days, feeding occurring two days prior to the harvest of RNA. In addition, early passage and senescent dermal fibroblasts were compared in deep quiescence (two weeks of culture in 0.5% FBS-containing medium and in subconfluent conditions normally consistent with log growth and in medium supplemented with 10% FBS). In addition, dermal fibroblasts from Hutchinson-Gilford Progeria Syndrome (HGPS) and age-matched normal controls were assayed synchronized in quiescence as described above for fetal and adult cells. Lastly, normal arm fibroblasts and iPS cells derived from said fibroblasts were included. As can be seen in FIGS. 2 and 3, Lamin B1 expression is highest in pluripotent stem cells, decreases progressively during fetal development during which time scarless regenerative potential is lost in the skin, and LMNB1 expression further declines in senescence and HGPS cells. Conversely, LMNA expression is relatively low in pluripotent stem cells, and increases in development and aging in vivo and potentially in vitro.

### Example 2: The Adult Markers COX7A1 and PCDHGA12 Correlate with CSC and EMT Markers in Diverse Cancer Types

RNA-sequencing of 1018 diverse cancer types including carcinomas, adenocarcinomas, sarcomas, blood cell leukemias and lymphomas, gliomas, pleural tumors, neuroblastomas, melanomas, as well as other cancer types. Sarcomas included osteosarcomas, chondrosarcomas, Ewing's sarcomas, liposarcomas, leiomyosarcomas, rhabdomyosarcomas, as well as others. Carcinomas and adenocarcinomas included those from the breast, lung, prostate, pancreas, colon, stomach, esophagus, as well as others.

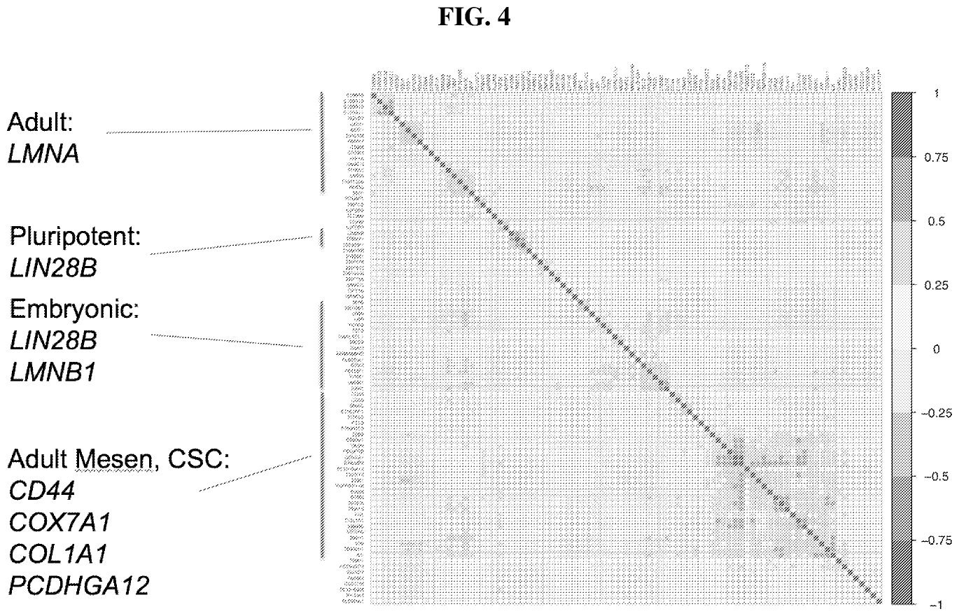

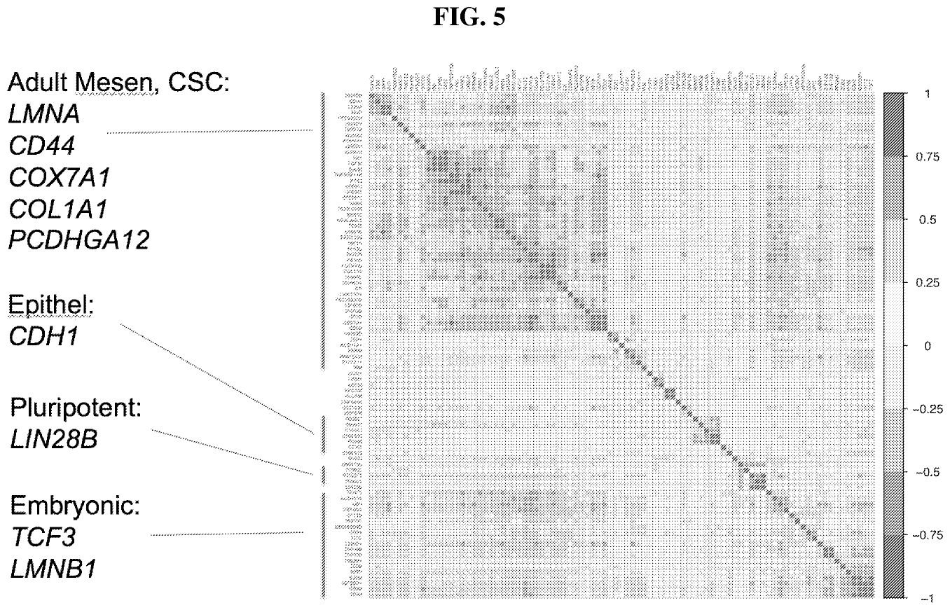

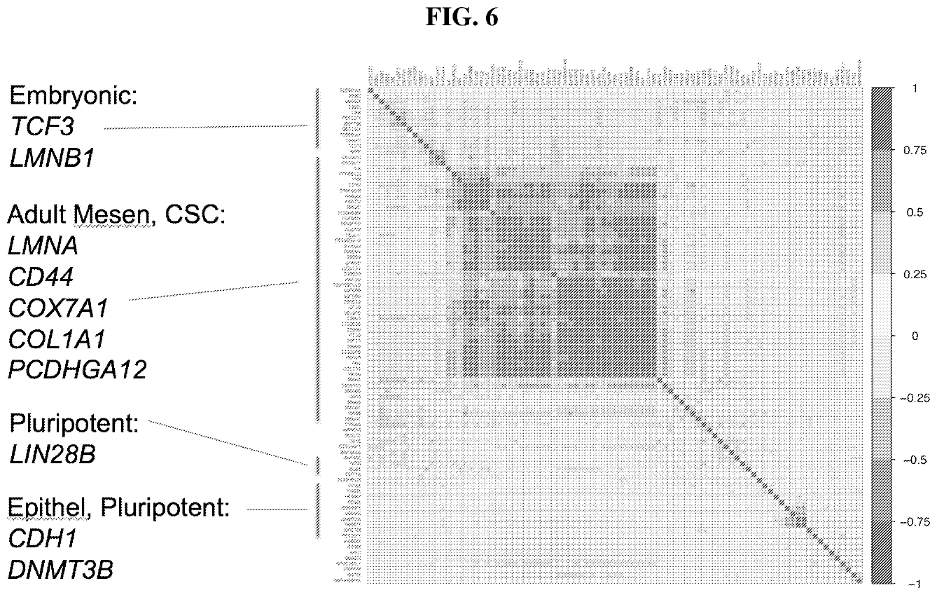

As shown in FIGS. 4, 5, and 6, LMNA expression correlated with CSC markers and EMT markers as opposed to embryonic or epithelial markers. In addition, LMNB1 and its receptor LBR correlated with cells not showing EMT markers (i.e. LMNA and LMNB1 were inversely correlated in cancer cell lines as they are in normal development).

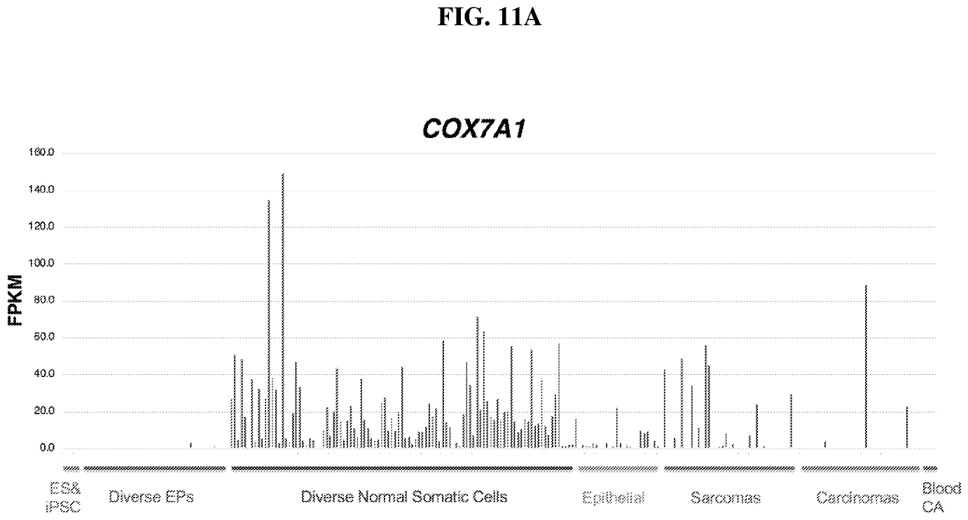

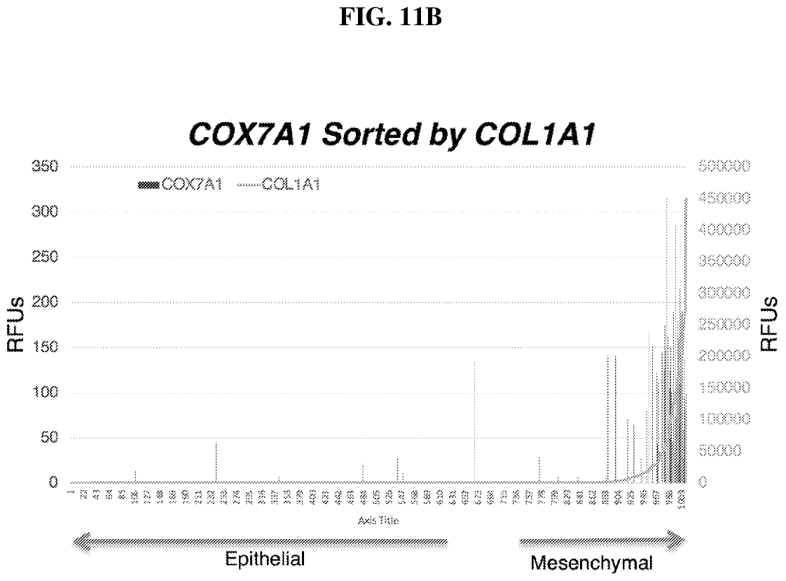

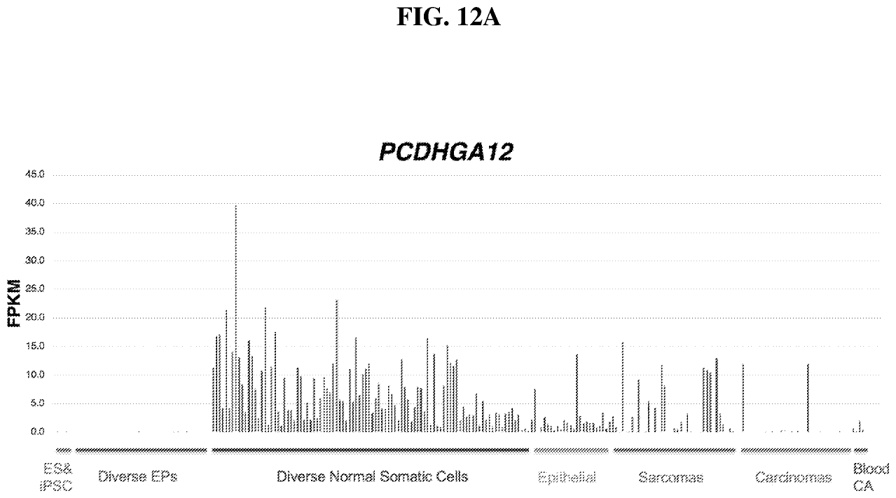

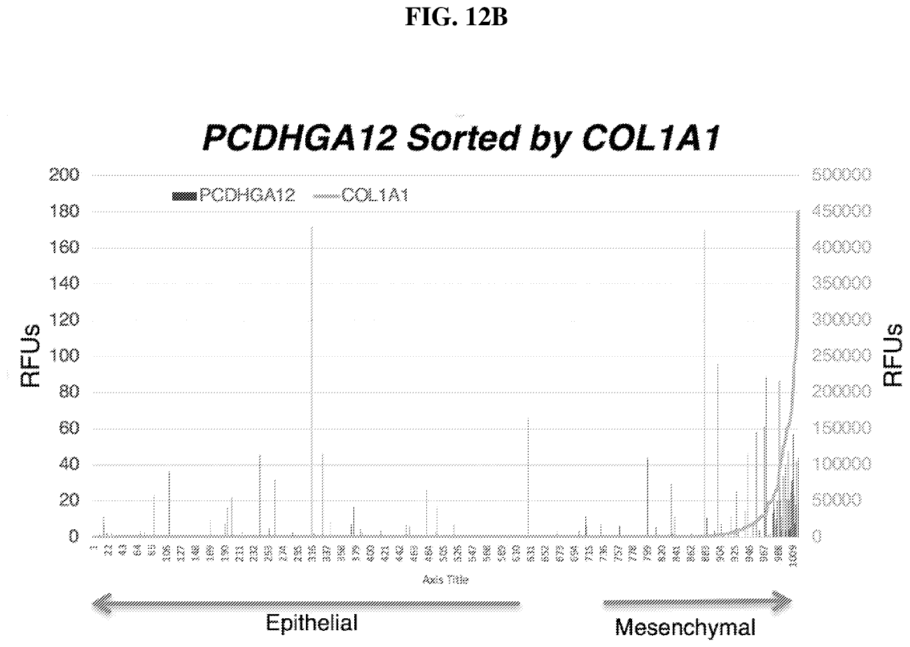

As shown in FIGS. 11 and 12, the expression of the adult markers COX7A1 and PCDHGA12 are not expressed or are expressed at relatively low levels in pluripotent stem cells and diverse hESC-derived clonal embryonic progenitor cells, but are expressed in adult stromal and epithelial cells. In the RNA-sequence data from 1018 diverse cancer cell lines referred to supra, the expression of COX7A1 and PCDHGA12 strongly correlated with the EMT marker COL1A1 as well as numerous other EMT and CSC markers (FIGS. 4-6 and data not shown).

### Example 3: Methods to Accelerate the Temporal Reprogramming of LMNA and LMNB1 Expression Utilizing TCF3 and POU2F1

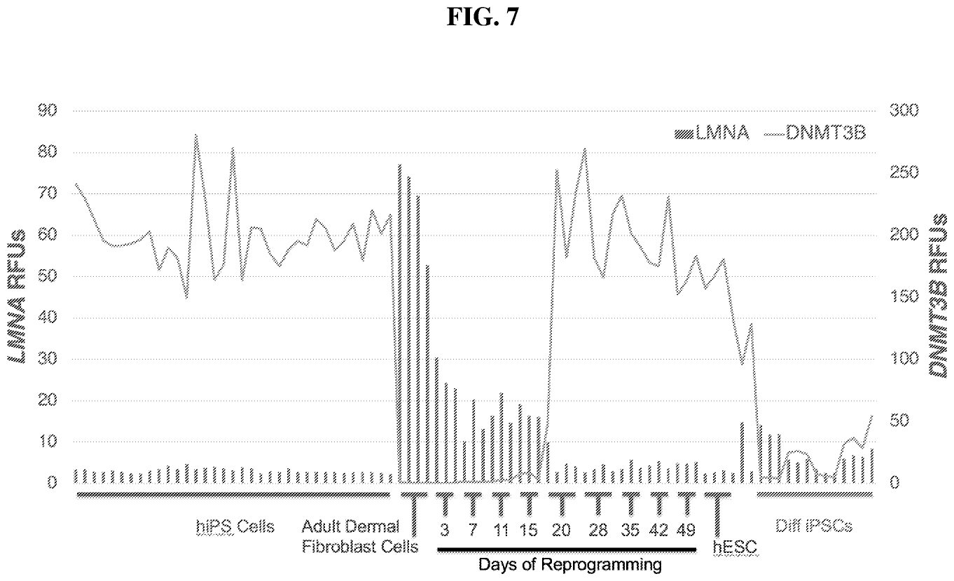

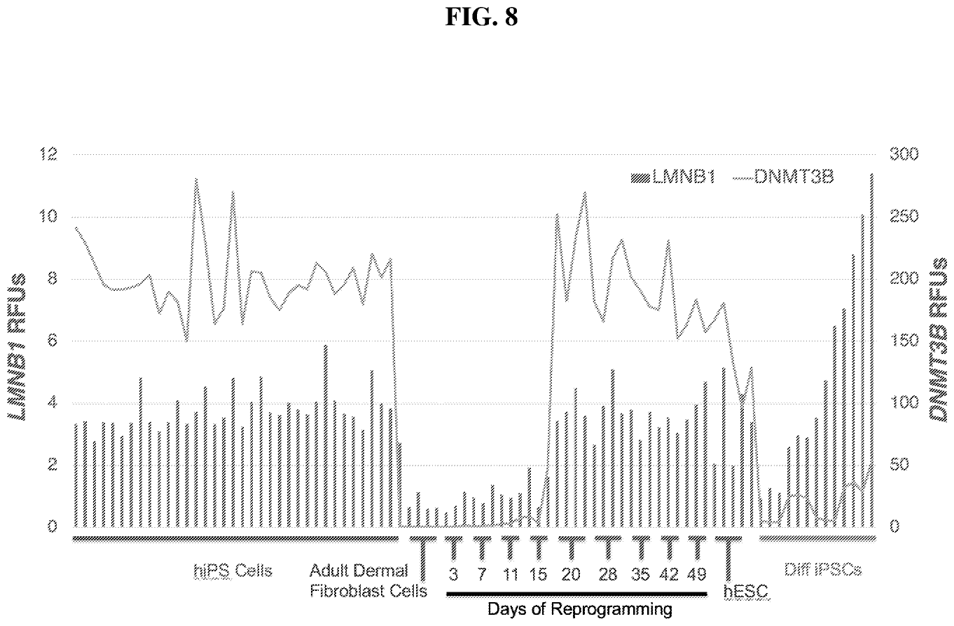

Since the goal of iTR and iS-CSC is to transiently express reprogramming factors and revert cells to a pre-EFT-like pattern of gene expression, and since, as disclosed herein, LMNA and LMNB1 expression are regulators of EFT, methods are necessary to reprogram LMNA and LMNB1 expression within 1-2 weeks to levels observed at or slightly before that shown herein for dermal fibroblasts at eight weeks of gestation and synchronized in medium as described herein. As shown in FIGS. 7, 8, 9, and 10, LMNA and LMNB1 show relatively slow responsiveness to KLF4, OCT4, SOX2, and MYC with full reprogramming only occurring after approximately 20 days of treatment which is also when pluripotency markers begin to be expressed. Since the goal of iTR and iS-CSC is to induce a pre-EFT, not a pluripotency phenotype, it would be useful to reprogram LMNA and LMNB1 expression between 1-2 weeks.

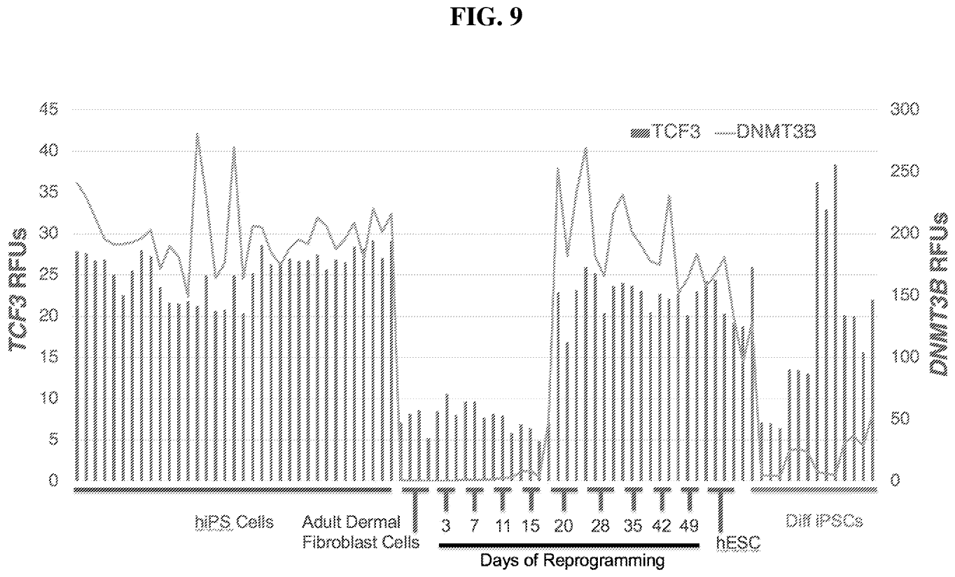

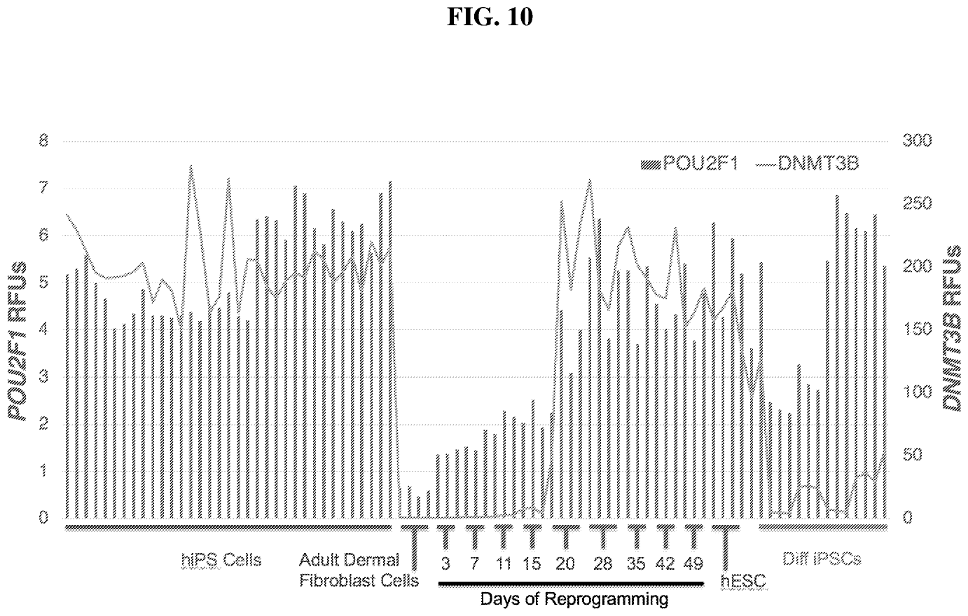

In the present invention we teach that TCF3 and POU2F1 are capable of reprogramming LMNA and LMNB1 expression. As shown in FIGS. 9 and 10, the reprogrammed expression of these factors is also normally delayed. We therefore disclose that the exogenous administration of TCF3 and POU2F1 by administration of RNA or DNA nucleotide sequences as described herein will accelerate LMNA and LMNB1 reprogramming and the efficiency of iTR and iS-CSC.

### Example 4: The Knockdown of LMNA in Adult Cells or the Knockdown of LMNB1 in Embryonic and Cancer Cells Alters the Regenerative Phenotype and Regenerative Gene Expression

LMNA and LMNB1 knockdown was performed in three human cell lines: MDW-1 (adult-derived normal dermal fibroblasts), 4D20.8 (hESC-derived clonal embryonic progenitors (pre-fetal) capable of osteochondral differentiation), and HT-1080 fibrosarcoma cells that exhibit a pre-EFT (pre-fetal) pattern of gene expression. The three cell lines were cultured in Dulbecco's Modified Eagle Medium (DMEM) supplemented with 10% fetal bovine serum (FBS, Hyclone), glutamate and sodium pyruvate in a fully humidified incubator containing 5% CO₂ at 37° C.

For the knockdown of LMNA and LMNB1, we purchased lentiviral particles from GeneCopoeia (Cat #: LPP-HSH010673-LVRU6GP and LPP-HSH088645-LVRU6GP) for transduction of shRNA. shRNA vector is driven by U6 pol III promoters with great knockdown efficiency and eGFP reporter genes for monitoring transduction efficiencies as well as allows stable cell selection with puromycin marker. The cells were seeded into 6-well cell culture plates at a concentration of 2×10⁵ cells/well. The following day, the cells were infected for 18 hours with the virus-containing supernatant at a multiplicity of infection (MOI) of 10 supplemented with 8.0 μg/ml polybrene.

To select stably transduced cells, the cells were cultured in the presence of 1.0-10 ug/ml puromycin. The knockdown cells were screened using RT-PCR to determine the levels of LMNA and LMNB1 expressions and monitored by checking the simultaneous co-expression of the eGFP reporter gene by fluorescence microscopy. Three individual shRNA constructs and a separate scrambled control shRNA were tested and selected the line with best knockdown efficiency.

Next, we performed scratch wound healing cell migration assays to demonstrate that alterations in the expression of LMNA or LMNB1 genes result in altered regenerative phenotype in normal cells or altered proliferation/migration of cancer cells. Cell proliferation and migration in normal or cancer cells is known to be modulated by numerous molecular pathways, however, as taught in the present disclosure, the role of LMNA and LMNB1 as pan-cell type regulators of the cell and tissue regeneration or cancer suppression is largely unknown.

We utilized an IncuCyte instrument migration assay to measure the effects of LMNA and LMNB1 knockdown in the three aforementioned cell lines. Cells were seeded to confluence with 2.0×10⁴ cells/well in a 96-well ImageLock™ plate in incubator with 5% CO₂ at 37° C. The next day, precise wounds were created by gently removing the cells from the confluent monolayer using an array of 96 pins.

After washing, media was added, and the plate was placed inside the IncuCyte by monitoring cells every 2 hours for 24 hours. For quantifying the cell migration over time, we used Relative Wound Density (RWD). This metric allows us measuring the spatial cell density in the wound area relative to the spatial cell density outside of the wound area at every time point. It is designed to be zero at t=0, and 100% when the cell density inside the wound is the same as the cell density outside the initial wound. In this respect, the metric is self-normalizing for changes in cell density which may occur outside the wound as a result of cell proliferation and/or pharmacological effects.

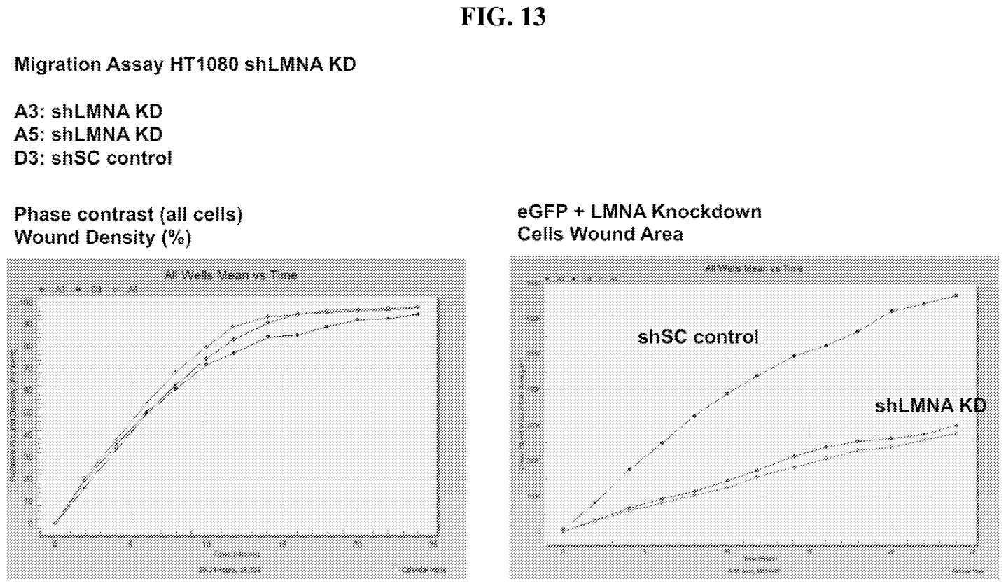

Proliferation of the adult-derived dermal fibroblasts with knockdown of LMNA but not LMNB1 can readily be measured as described. Similarly, effects of knockdown of LMNA but not LMNB1 in cancer cells such as HT1080 can be assayed. As shown in FIG. 13, the knockdown of LMNA (shLMNA KD) resulted in marked decreased migration of the cancer cell line HT1080 consistent with the reduction of the adult-like, CSC, EMT phenotype that is characterized by migration. Therefore, reduction in LMNA levels by siRNA or other means that reduce LMNA levels reduces migration and metastasis in cancer cells.

Using Quantitative real-time PCR, the gene expression of adult markers such as COX7A1 and PCDHGA12 can readily be assayed to observe the reprogramming and induction of the regenerative phenotype in adult-derived cells such as adult-derived dermal fibroblasts and increased proliferation of cancer cells following knockdown of LMNA, and the reduced proliferation and migration of embryonic (pre-fetal) cells such as the aforementioned clonal embryonic progenitor cell line 4D20.8 or the cancer cell line HT-1080 following knockdown of LMNB1 expression.

RNA is isolated with the RNeasy kit (Qiagen, Valencia, CA, USA). First-strand cDNA was primed with oligo (dT) primers, and qPCR was performed with TaqMan primer sets (Life

Technologies-Applied Biosystems, Foster City, CA, USA). Relative expression levels is normalized to GAPDH and 18s and calculated by the 2\hat{ } dCt method.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

### INCORPORATION BY REFERENCE

All publications and patents cited in this specification are herein incorporated by reference as if each individual publication or patent were specifically and individually indicated to be incorporated by reference and are incorporated herein by reference in their entirety to disclose and describe the methods and/or materials in connection with which the publications are cited. The citation of any publication is for its disclosure prior to the filing date and should not be construed as an admission that the present invention is not entitled to antedate such publication by virtue of prior invention.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### REFERENCES

1. Grunwald, G. B. The conceptual and experimental foundations of vertebrate embryonic cell adhesion research. Dev Biol (N Y 1985) 7, 129-158 (1991).
2. Steinberg, M. S. & Gilbert, S. F. Townes and Holtfreter (1955): directed movements and selective adhesion of embryonic amphibian cells. J Exp Zool A Comp Exp Biol 301, 701-706 (2004).
3. Wilson, H. V. On Some Phenomena of Coalescence and Regeneration in Sponges. J. Exp. Zool. 5, 245-258 (1907).
4. Spemann, H. & Mangold, H. Induction of embryonic primordia by implantation of organizers from a different species. 1923. Int J Dev Biol 45, 13-38 (2001).
5. Moscona, A. & Moscona, H. The dissociation and aggregation of cells from organ rudiments of the early chick embryo. J Anat 86, 287-301 (1952).
6. Li, G. X., Moscona, M. H. & Moscona, A. A. Effect of embryonic age on aggregability, histogenesis and biochemical differentiation in the embryonic chick and quail neural retina.

Sci Sin B 27, 371-379 (1984).
7. Williams, G. Pleiotropy, Natural Selection, and the Evolution of Senescence. Evolution 11, 398-411 (1957).
8. Kim, N. W. et al. Specific association of human telomerase activity with immortal cells and cancer. Science 266, 2011-2015 (1994).
9. Dreyer, W. J. G., W. R.; Hood, L. The Genetic, Molecular, and Cellular Basis of Antibody Formation: Some Facts and a Unifying Hypothesis. Cold Spring Harb Symp Quant Biol 32, 353-367 (1967).
10. Dreyer, W. J. & Roman-Dreyer, J. Cell-surface area codes: mobile-element related gene switches generate precise and heritable cell-surface displays of address molecules that are used for constructing embryos. Genetica 107, 249-259 (1999).
11. Wu, Q. & Maniatis, T. A striking organization of a large family of human neural cadherin-like cell adhesion genes. Cell 97, 779-790 (1999).
12. Thu, C. A. et al. Single-cell identity generated by combinatorial homophilic interactions between alpha, beta, and gamma protocadherins. Cell 158, 1045-1059 (2014).
13. Schreiner, D. & Weiner, J. A. Combinatorial homophilic interaction between gamma-protocadherin multimers greatly expands the molecular diversity of cell adhesion. Proc Natl Acad Sci USA 107, 14893-14898 (2010).
14. Kaneko, R. et al. Allelic gene regulation of Pcdh-alpha and Pcdh-gamma clusters involving both monoallelic and biallelic expression in single Purkinje cells. J Biol Chem 281, 30551-30560 (2006).
15. Lefebvre, J. L., Kostadinov, D., Chen, W. V., Maniatis, T. & Sanes, J. R. Protocadherins mediate dendritic self-avoidance in the mammalian nervous system. Nature 488, 517-521 (2012).
16. Halbleib, J. M. & Nelson, W. J. Cadherins in development: cell adhesion, sorting, and tissue morphogenesis. Genes & development 20, 3199-3214 (2006).
17. Thomson, J. A. et al. Embryonic stem cell lines derived from human blastocysts. Science 282, 1145-1147 (1998).
18. Clevers, H. Modeling Development and Disease with Organoids. Cell 165, 1586-1597 (2016).
19. West, M. D. et al. Use of deep neural network ensembles to identify embryonic-fetal transition markers: repression of COX7A1 in embryonic and cancer cells. Oncotarget 9, 7796-7811 (2018).
20. West, M. D. et al. The ACTCellerate initiative: large-scale combinatorial cloning of novel human embryonic stem cell derivatives. Regen Med 3, 287-308 (2008).
21. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Methods 10, 1213-1218 (2013).
22. Yang, J. & Corces, V. G. Insulators, long-range interactions, and genome function. Curr Opin Genet Dev 22, 86-92 (2012).
23. Sternberg, H. et al. A human embryonic stem cell-derived clonal progenitor cell line with chondrogenic potential and markers of craniofacial mesenchyme. Regen Med 7, 481-501 (2012).
24. West, M. D. et al. Clonal derivation of white and brown adipocyte progenitor cell lines from human pluripotent stem cells. Stem cell research & therapy 10, 7 (2019).
25. Toyota, M. et al. CpG island methylator phenotype in colorectal cancer. Proc Natl Acad Sci USA 96, 8681-8686 (1999).
26. Ogino, S. et al. Evaluation of markers for CpG island methylator phenotype (CIMP) in colorectal cancer by a large population-based sample. J Mol Diagn 9, 305-314 (2007).
27. Lund, E. G., Duband-Goulet, I., Oldenburg, A., Buendia, B. & Collas, P. Distinct features of lamin A-interacting chromatin domains mapped by ChIP-sequencing from sonicated or micrococcal nuclease-digested chromatin. Nucleus 6, 30-39 (2015).
28. Guelen, L. et al. Domain organization of human chromosomes revealed by mapping of nuclear lamina interactions. Nature 453, 948-951 (2008).
29. Khan, A. & Zhang, X. dbSUPER: a database of super-enhancers in mouse and human genome. Nucleic Acids Res 44, D164-171 (2016).
30. Kim, S., Yu, N. K. & Kaang, B. K. CTCF as a multifunctional protein in genome regulation and gene expression. Exp Mol Med 47, e166 (2015).
31. Igolkina, A. A. et al. H3K4me3, H3K9ac, H3K27ac, H3K27me3 and H3K9me3 Histone Tags Suggest Distinct Regulatory Evolution of Open and Condensed Chromatin Landmarks. Cells 8 (2019).
32. Goodman, K. M. et al. Protocadherin cis-dimer architecture and recognition unit diversity. Proc Natl Acad Sci USA 114, E9829-E9837 (2017).
33. Kehayova, P., Monahan, K., Chen, W. & Maniatis, T. Regulatory elements required for the activation and repression of the protocadherin-alpha gene cluster. Proc Natl Acad Sci USA 108, 17195-17200 (2011).
34. Sternberg, H. et al. Seven diverse human embryonic stem cell-derived chondrogenic clonal embryonic progenitor cell lines display site-specific cell fates. Regen Med (2012).
35. Jakab, K. et al. Tissue engineering by self-assembly and bio-printing of living cells. Biofabrication 2, 022001 (2010).
36. Constantinescu, D., Gray, H. L., Sammak, P. J., Schatten, G. P. & Csoka, A. B. Lamin A/C expression is a marker of mouse and human embryonic stem cell differentiation. Stem Cells 24, 177-185 (2006).
37. Shimi, T. et al. The role of nuclear lamin B1 in cell proliferation and senescence. Genes & development 25, 2579-2593 (2011).
38. West, M. D. et al. Toward a unified theory of aging and regeneration. Regen Med 14, 867-886 (2019).
39. Sternberg, H., Janus, J. & West, M. D. Defining cell-matrix combination products in the era of pluripotency. Biomatter 3 (2013).
40. Almenar-Queralt, A. et al. Chromatin establishes an immature version of neuronal protocadherin selection during the naive-to-primed conversion of pluripotent stem cells. Nat Genet 51, 1691-1701 (2019).
41. El Hajj, N., Dittrich, M. & Haaf, T. Epigenetic dysregulation of protocadherins in human disease. Semin Cell Dev Biol 69, 172-182 (2017).
42. Kaczkowski, B. et al. Transcriptome Analysis of Recurrently Deregulated Genes across Multiple Cancers Identifies New Pan-Cancer Biomarkers. Cancer research 76, 216-226 (2016).
43. K., V. R. L. Cellular Pathology (Originally Published 1859). (Classics of Medicine, U.S.A.; 1978).
44. Warburg, O. On the origin of cancer cells. Science 123, 309-314 (1956).
45. Manzo, G. Similarities Between Embryo Development and Cancer Process Suggest New Strategies for Research and Therapy of Tumors: A New Point of View. Front Cell Dev Biol 7, 20 (2019).
46. Abe, M. et al. CpG island methylator phenotype is a strong determinant of poor prognosis in neuroblastomas. Cancer research 65, 828-834 (2005).
47. Henrich, K. O. et al. Integrative Genome-Scale Analysis Identifies Epigenetic Mechanisms of Transcriptional Deregulation in Unfavorable Neuroblastomas. Cancer research 76, 5523-5537 (2016).
48. Vega-Benedetti, A. F. et al. Clustered protocadherins methylation alterations in cancer. Clin Epigenetics 11, 100 (2019).
49. West, M. D. et al. The germline/soma dichotomy: implications for aging and degenerative disease. Regen Med 11, 331-334 (2016).
50. Rober, R. A., Weber, K. & Osborn, M. Differential timing of nuclear lamin A/C expression in the various organs of the mouse embryo and the young animal: a developmental study. Development 105, 365-378 (1989).
51. Solovei, I. et al. LBR and lamin A/C sequentially tether peripheral heterochromatin and inversely regulate differentiation. Cell 152, 584-598 (2013).
52. Tardat, M. & Dejardin, J. Telomere chromatin establishment and its maintenance during mammalian development. Chromosoma 127, 3-18 (2018).
53. van Steensel, B. & Belmont, A. S. Lamina-Associated Domains: Links with Chromosome Architecture, Heterochromatin, and Gene Repression. Cell 169, 780-791 (2017).
54. Dixon, J. R. et al. Chromatin architecture reorganization during stem cell differentiation. Nature 518, 331-336 (2015).
55. McClay, J. L. et al. A methylome-wide study of aging using massively parallel sequencing of the methyl-CpG-enriched genomic fraction from blood in over 700 subjects. Hum Mol Genet 23, 1175-1185 (2014).
56. Salpea, P. et al. Postnatal development- and age-related changes in DNA-methylation patterns in the human genome. Nucleic Acids Res 40, 6477-6494 (2012).
57. Mah, K. M., Houston, D. W. & Weiner, J. A. The gamma-Protocadherin-C3 isoform inhibits canonical Wnt signalling by binding to and stabilizing Axin1 at the membrane. Sci Rep 6, 31665 (2016).
58. Ocampo, A. et al. In Vivo Amelioration of Age-Associated Hallmarks by Partial Reprogramming. Cell 167, 1719-1733 e1712 (2016).
59. Lanza, R. P., Cibelli, J. B. & West, M. D. Human therapeutic cloning. Nat Med 5, 975-977 (1999).
60. Plath, K. & Lowry, W. E. Progress in understanding reprogramming to the induced pluripotent state. Nat Rev Genet 12, 253-265 (2011).
61. Soufi, A., Donahue, G. & Zaret, K. S. Facilitators and impediments of the pluripotency reprogramming factors' initial engagement with the genome. Cell 151, 994-1004 (2012).
62. Trapnell, C. et al. Differential gene and transcript expression analysis of RNA-seq experiments with TopHat and Cufflinks. Nat Protoc 7, 562-578 (2012).
63. Corces, M. R. et al. An improved ATAC-seq protocol reduces background and enables interrogation of frozen tissues. Nat Methods 14, 959-962 (2017).
64. Bentsen, M. et al. ATAC-seq footprinting unravels kinetics of transcription factor binding during zygotic genome activation. Nat Commun 11, 4267 (2020).
65. Zhang, Y. et al. Model-based analysis of ChIP-Seq (MACS). Genome Biol 9, R137 (2008).
66. Fornes, O. et al. JASPAR 2020: update of the open-access database of transcription factor binding profiles. Nucleic Acids Res 48, D87-D92 (2020).
67. Robinson, J. T. et al. Integrative genomics viewer. Nat Biotechnol 29, 24-26 (2011).
68. Krueger, F. & Andrews, S. R. Bismark: a flexible aligner and methylation caller for Bisulfite-Seq applications. Bioinformatics 27, 1571-1572 (2011).
69. Juhling, F. et al. metilene: fast and sensitive calling of differentially methylated regions from bisulfite sequencing data. Genome Res 26, 256-262 (2016).
70. Mahmoudi, S., and Brunet, A. (2012) Aging and reprogramming: a two-way street. Curr Opin Cell Biol 24, 744-756
71. Lapasset, L., Milhavet, O., Prieur, A., Besnard, E., Babled, A., Ait-Hamou, N., Leschik, J., Pellestor, F., Ramirez, J. M., De Vos, J., Lehmann, S., and Lemaitre, J. M. (2011) Rejuvenating senescent and centenarian human cells by reprogramming through the pluripotent state. Genes Dev 25, 2248-2253
72. Ocampo, A., Reddy, P., Martinez-Redondo, P., Platero-Luengo, A., Hatanaka, F., Hishida, T., Li, M., Lam, D., Kurita, M., Beyret, E., Araoka, T., Vazquez-Ferrer, E., Donoso, D., Roman, J. L., Xu, J., Rodriguez Esteban, C., Nunez, G., Nunez Delicado, E., Campistol, J. M., Guillen, I., Guillen, P., and Izpisua Belmonte, J. C. (2016) In Vivo Amelioration of Age-Associated Hallmarks by Partial Reprogramming. Cell 167, 1719-1733 e1712
73. West, M. D., Labat, I., Sternberg, H., Larocca, D. , Nasonkin, I., Chapman, K., Singh, R., Larocca, J., Makarev, E., Aliper, A. M., Kazennov, A., Alekseenko, A., Shuvalov, N., Cheskidova, E., Alekseev, A., Artemov, A., Putin, E., Mamoshina, P., Nikita P., Copeland, K., Korzinkin, M., Lezhnina, K., Izumchenko, E., Zhavoronkov, A. (2017) Use of Deep Neural Network Ensembles to Identify Embryonic-Fetal Transition Markers: Repression of COX7A1 in Embryonic and Cancer Cells. Oncotarget
74. Zhou, W., Choi, M., Margineantu, D., Margaretha, L., Hesson, J., Cavanaugh, C., Blau, C. A., Horwitz, M. S., Hockenbery, D., Ware, C., and Ruohola-Baker, H. (2012) HIFlalpha induced switch from bivalent to exclusively glycolytic metabolism during ESC-to-EpiSC/hESC transition. EMBO J 31, 2103-2116
75. Mishra, N., Timilsina, U., Ghimire, D., Dubey, R. C., and Gaur, R. (2017) Downregulation of cytochrome c oxidase subunit 7A1 expression is important in enhancing cell proliferation in adenocarcinoma cells. Biochem Biophys Res Commun 482, 713-719
76. West, M. D., Sargent, R. G., Long, J., Brown, C., Chu, J. S., Kessler, S., Derugin, N., Sampathkumar, J., Burrows, C., Vaziri, H., Williams, R., Chapman, K. B., Larocca, D., Loring, J. F., and Murai, J. (2008) The ACTCellerate initiative: large-scale combinatorial cloning of novel human embryonic stem cell derivatives. Regen Med 3, 287-308
77. West, M. D., Chang, C. F., Larocca, D., Li, J., Jiang, J., Sim, P., Labat, I., Chapman, K. B., Wong, K. E., Nicoll, J., Van Kanegan, M. J., de Grey, A., Nasonkin, I. O., Stahl, A., and Sternberg, H. (2019) Clonal derivation of white and brown adipocyte progenitor cell lines from human pluripotent stem cells. Stem Cell Res Ther 10, 7
78. Ohnuki, M., Tanabe, K., Sutou, K., Teramoto, I., Sawamura, Y., Narita, M., Nakamura, M., Tokunaga, Y., Nakamura, M., Watanabe, A., Yamanaka, S., and Takahashi, K. (2014) Dynamic regulation of human endogenous retroviruses mediates factor-induced reprogramming and differentiation potential. Proc Natl Acad Sci USA 111, 12426-12431
79. Olova, N., Simpson, D. J., Marioni, R. E., and Chandra, T. (2019) Partial reprogramming induces a steady decline in epigenetic age before loss of somatic identity. Aging Cell 18, e12877
80. Tanabe, K., Nakamura, M., Narita, M., Takahashi, K., and Yamanaka, S. (2013) Maturation, not initiation, is the major roadblock during reprogramming toward pluripotency from human fibroblasts. Proc Natl Acad Sci USA 110, 12172-12179
81. Kurita, M., Araoka, T., Hishida, T., O'Keefe, D. D., Takahashi, Y., Sakamoto, A., Sakurai, M., Suzuki, K., Wu, J., Yamamoto, M., Hernandez-Benitez, R., Ocampo, A., Reddy, P., Shokhirev, M. N., Magistretti, P., Nunez Delicado, E., Eto, H., Harii, K., and Izpisua Belmonte, J. C. (2018) In vivo reprogramming of wound-resident cells generates skin epithelial tissue. Nature 561, 243-247
82. Tang, Y., and Cheng, L. (2017) Cocktail of chemical compounds robustly promoting cell reprogramming protects liver against acute injury. Protein Cell 8, 273-283
83. Marion, R. M., and Blasco, M. A. (2017) Long Live Partial Reprogramming. Circ Res 120, 1381-1383
84. Tamanini, S., Comi, G. P., and Corti, S. (2018) In Vivo Transient and Partial Cell Reprogramming to Pluripotency as a Therapeutic Tool for Neurodegenerative Diseases. Mol Neurobiol 55, 6850-6862
85. Beyret, E., Martinez Redondo, P., Platero Luengo, A., and Izpisua Belmonte, J. C. (2018) Elixir of Life: Thwarting Aging With Regenerative Reprogramming. Circ Res 122, 128-141
86. Doeser, M. C., Scholer, H. R., and Wu, G. (2018) Reduction of Fibrosis and Scar Formation by Partial Reprogramming In Vivo. Stem Cells 36, 1216-1225

1. A method for inducing tissue regeneration in a target cell, a method comprising the steps:
1) contacting the target cell with an agent that increases the ratio of lamin B1 protein compared to lamin A protein in the target cell; and
2) contacting the target cell with one or more nucleic acids encoding TERT, wherein TERT is transiently expressed to increase telomere length in the target cells, thereby inducing tissue regeneration.

2. The method of claim 1, wherein the agent that increasing the ratio of lamin B1 protein compared to lamin A protein is one or more nucleic acids encoding LMNB1, thereby increasing lamin B1 protein.

3. The method of claim 1, wherein the agent that increasing the ratio of lamin B1 protein compared to lamin A protein is an siRNA targeting LMNA mRNA, thereby reducing lamin A protein.

4. The method of claim 1, wherein the target cell is mammalian.

5. The method of claim 4, wherein the mammalian cell is non-human.

6. The method of claim 4, wherein the mammalian cell is human.

7. A method for inducing senolysis in cancer cells in a subject, a method comprising the steps:
1) contacting the cancer cell with an agent that increases the ratio of lamin B1 protein compared to lamin A protein in the cancer cell; and
2) contacting the cancer cells with an agent that induces apoptosis in cells with DNA damage.

8. The method of claim 7, wherein the agent that increasing the ratio of lamin B1 protein compared to lamin A protein is one or more nucleic acids encoding TCF3, thereby increasing lamin B1 protein.

9. The method of claim 7, wherein the agent that increasing the ratio of lamin B1 protein compared to lamin A protein is an siRNA targeting LMNA mRNA, thereiny reducing lamin A protein.

10. The method of claim 7, wherein the cancer cell is mammalian.

11. The method of claim 10, wherein the mammalian cell is non-human.

12. The method of claim 10, wherein the mammalian cell is human.

13. The method of and one of claims 7-12, wherein the apoptosis-inducing agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, BSI-201, or a combination thereof.

14. A method for inducing senolysis in cancer cells in a subject, a method comprising the steps:
1) contacting the cancer stem cells iPSC with one or more reprogramming factors selected from OCT4, SOX2, KLF4, NANOG, ESRRB, NR5A2, CEBPA, MYC, SALL4, LIN28A, and LIN28B without reprogramming the cells to pluripotency, wherein the expression of the one or more reprogramming factors is transient; and
2) contacting the cancer cells with an agent that induces apoptosis in cells with DNA damage.

15. The method of claim 14, wherein step (1) is contacting the cancer cells with one or more nucleic acids comprising a gene sequence encoding one or more of OCT4, SOX2, KLF4, NANOG, ESRRB, NR5A2, CEBPA, MYC, SALL4, LIN28A, and LIN28B, wherein the one or more nucleic acids expresses the one or more of OCT4, SOX2, KLF4, NANOG, ESRRB, NR5A2, CEBPA, MYC, SALL4, LIN28A, and LIN28B in the cancer cell.

16. The method of claim 14, wherein the reprogramming factors are SOX2, OCT4, and KLF4.

17. The method of claim 14, wherein the reprogramming factors are LIN28B, SOX2, NANOG, and OCT4.

18. The method of claim 14, wherein the cancer cell is mammalian.

19. The method of claim 18, wherein the mammalian cell is non-human.

20. The method of claim 18, wherein the mammalian cell is human.

21. The method of any on of claims 14-20, wherein the apoptosis-inducing agent is high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, BSI-201, or a combination thereof.


SEQUENCE LISTING (1 sequence)

SEQ ID NO: 1 - protein, 6 aa, Artificial Sequence
  Description of Artificial Sequence Synthetic 6xHis tag
HHHHHH